(19) United States
(12) Patent Application Publication
(10) Pub. No.: US 2006/0019356 A1
(43) Pub. Date: Jan. 26, 2006

(54) Polynucleotides encoding polypeptides involved in intermediates metabolism of the central metabolic pathway in Methylophilus methylotrophus

(76) Inventors: Yoshihiro Usuda, Kawasaki-shi (JP); Yousuke Nishio, Kawasaki-shi (JP); Hisashi Yasueda, Kawasaki-shi (JP); Shinichi Sugimoto, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/216,267

(22) Filed: Sep. 1, 2005

Related U.S. Application Data
(63) Continuation-in-part of application No. 10/375,266, filed on Feb. 28, 2003.

(51) Int. Cl. C07H 21/04; C12P 13/04; C12P 13/08; C12N 15/74

(52) U.S. Cl. 435/106; 435/115; 435/252.3; 435/471; 536/23.2

(57) ABSTRACT

The present invention provides polypeptides and polynucleotides involved in central intermediates metabolism in Methylophilus methylotrophus and methods of producing amino acids in microorganisms having enhanced or attenuated expression of these polypeptides and/or polynucleotides.

This application is a continuation-in-part under 35 U.S.C. §120 of Ser. No. 10/375,266, filed Feb. 28, 2003, the entirety of which is incorporated by reference.

### BACKGROUND OF THE INVENTION

1. Field of the Invention

The present invention relates to novel polynucleotides encoding proteins involved in intermediates metabolism of central metabolic pathway, derived from microorganisms belonging to methylotrophic bacteria and fragments thereof, polypeptides encoded by the polynucleotides and fragments thereof, polynucleotide arrays comprising the polynucleotides and fragments thereof.

2. Discussion of the Background

Amino acids such as L-lysine, L-glutamic acid, L-threonine, L-leucine, L-isoleucine, L-valine, and L-phenylalanine are industrially produced by fermentation by using microorganisms that belong to the genus Brevibacterium, Corynebacterium, Bacillus, Escherichia, Streptomyces, Pseudomonas, Arthrobacter, Serratia, Penicillium, Candida or the like. In order to improve the productivity of amino acids, strains of the aforementioned microorganisms that have been isolated from nature or artificial mutants thereof have been used. Various techniques have also been disclosed for enhancing activities of L-amino acid biosynthetic enzymes by using recombinant DNA techniques to increase the L-amino acid-producing ability.

L-amino acid production has been increased considerably by breeding of microorganisms such as those mentioned above and by improvements in production methods. However, in order to meet a future increase in the demand for L-amino acids, development of methods for more efficiently producing L-amino acids at lower cost are still desired.

Conventional methods for producing amino acids by fermentation using methanol, which is a raw fermentation material available in large quantities at a low cost, employ Achromobacter or Pseudomonas microorganisms (Japanese Patent Publication (Kokoku) No. 45-25273/1970), Protaminobacter microorganisms (Japanese Patent Application Laid-open (Kokai) No. 49-125590/1974), Protaminobacter or Methanomonas microorganisms (Japanese Patent Application Laid-open (Kokai) No. 50-25790/1975), Microcyclus microorganisms(Japanese Patent Application Laid-open (Kokai) No.52-18886/1977), Methylobacillus microorganisms(Japanese Patent Application Laid-open (Kokai) No. 4-91793/1992), Bacillus microorganisms(Japanese Patent Application Laid-open (Kokai) No. 3-505284/1991) and others.

However, only a few methods have been described for producing L-amino acids using Methylophilus bacteria in conjunction with recombinant DNA technology. Although methods described in EP 0 035 831 A, EP 0 037 273 A, and EP 0 066 994 A have been described as methods for transforming Methylophilus bacteria using recombinant DNA, applying recombinant DNA techniques to improvement of amino acid productivity of Methylophilus bacteria has not been described. Only WO 00/61723 and WO 02/38777 disclose the improved production of lysine and phenylalanine, respectively, using genes involved in each amino acid biosynthesis.

Therefore, prior to the present invention genes isolated from Methylophilus bacteria that are involved in intermediates metabolism of central metabolic pathway and which can be used to improve the yield of amino acids in cultured microorganisms remain elusive and undisclosed.

### SUMMARY OF THE INVENTION

An object of the present invention is to provide novel measures for the improved production of amino acids or an amino acid, where these amino acids include asparagine, threonine, serine, glutamate, glycine, alanine, cysteine, valine, methionine, isoleucine, leucine, tyrosine, phenylalanine, histidine, lysine, tryptophan, arginine, and the salts thereof. In a preferred embodiment the amino acids are L-amino acids.

Such a process includes bacteria, which express a protein comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, and SEQ ID NO:42.

In one embodiment the polypeptides are encoded by a polynucleotide selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, and SEQ ID NO:41. In another embodiment the polypeptides are encoded by other polynucleotides which have substantial identity to the herein described polynucleotides or those which hybridize under stringent conditions.

Another object of the invention is to provide polynucleotide sequences selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, and SEQ ID NO:41; as well as those polynucleotides that have substantial identity to these nucleotide sequences, preferably at least 95% identity.

Another object of the invention is to provide isolated polypeptides having a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, and SEQ ID NO:42; as well as those polypeptides that have substantial identity to these amino acid sequences, preferably at least 95% identity.

A further object of the invention is a method for producing a protein or proteins by culturing host cells containing the herein described polynucleotides under conditions and for a time suitable for expression of the protein and collecting the protein produced thereby.

Another object is the use of host cells having the polynucleotides described herein to produce amino acids, as well as the use of such isolated polypeptides in the production of amino acids.

Other objects of the invention include methods of detecting nucleic acid sequences homologous to at least one of: SEQ ID NO: I, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, and SEQ ID NO:41, particularly nucleic acid sequences encoding polypeptides that herein described proteins or polypeptides and methods of making nucleic acids encoding such polypeptides.

The above objects highlight certain aspects of the invention. Additional objects, aspects and embodiments of the invention are found in the following detailed description of the invention.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

Methylophilus methylotrophus (M. methylotrophus) is a gram negative ribulose monophosphate cycle methanol-utilizer, which can be used for the large-scale production of a variety of fine chemicals including amino acids, nucleic acids, vitamins, saccharides, and so on. The polynucleotides of this invention, therefore, can be used to identify microorganisms, which can be used to produce fine chemicals, for example, by fermentative processes. Modulation of the expression of the polynucleotides encoding enzymes which are involved in metabolism of central intermediates in central metabolic pathway of the present invention, can be used to modulate the production of one or more fine chemicals from a microorganism (e.g., to improve the yield of production of one or more fine chemicals from Methylophilus or Methylbacillus species).

The proteins encoded by the polynucleotides of the present invention are capable of, for example, performing a function involved in the metabolism of central intermediates in M. methylotrophus, such as fructose 6-phosphate, glucose 6-phosphate, 6-phosphoglucono-1,5-lactone, 6-phosphogluconate, 2-dehydro-3-deoxy-gluconate 6-phosphate, glyceraldehyde 3-phosphate, dihydroxyacetone phosphate, fructose 1,6-bisphosphate, erythrose 4-phosphate, sedoheptulose 7-phosphate, xylulose 5-phosphate, ribose 5-phosphate, ribulose 5-phosphate, glycerate-3-phosphate, glycerate-2-phosphate, or phosphoenolpyruvate.

Given the availability of cloning vectors used in M. methylotrophus, such as those disclosed in Methane and Methanol Utilizers, Plenum Press, New York (1992) edited by J. Colin Murrell and Howard Dalton, the nucleic acid molecules of the present invention may be used in the genetic engineering of this organism to make it a better or more efficient producer of one or more fine chemicals.

There are a number of mechanisms by which the alteration of a protein of the present invention may affect the yield, production, and/or efficiency of production of a fine chemical from M. methylotrophus bacteria, which have the altered protein incorporated. Improving the ability of the cell to synthesize pyruvate (e.g., by manipulating the genes encoding enzymes involved in the conversion of 6-phosphogluconate into pyruvate), one may increase the yield or productivity of desired fine chemicals. Furthermore, by suppressing the activity of enzymes involved in the wasteful pathway such as the conversion of 6-phosphogluconate to ribulose 5-phosphate and carbon dioxide, one may also increase the yield or productivity of desired fine chemicals.

“L-amino acids” or “amino acids” as used herein means one or more amino acids, including their salts, preferably chosen from the following: L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, and L-arginine.

“Isolated” as used herein means separated out of its natural environment.

“Substantial identity” as used herein refers to polynucleotides and polypeptides which are at least 70%, preferably at least 80% and more preferably at least 90% to 95% identical to the polynucleotides and polypeptides, respectively, according to the present invention.

“Polynucleotide” as used herein relates to polyribonucleotides and polydeoxyribonucleotides, it being possible for these to be non-modified RNA or DNA or modified RNA or DNA.

“Polypeptides” as used herein are understood to mean peptides or proteins which comprise two or more amino acids bonded via peptide bonds. In particular, the term refers to polypeptides which are at least 70%, preferably at least 80% and more preferably at least 90% to 95% identical to the polypeptides according to the present invention. Included within the scope of the present invention are polypeptide fragments of the polypeptides having a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, and SEQ ID NO:42 or those which are identical to those described herein.

“Polynucleotides which encode the polypeptide” of the invention as used herein is understood to mean the sequences exemplified in this application as well as those sequences which have substantial identity to the nucleic acid sequences at least one of: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, and SEQ ID NO:41 and which encode a molecule having one or more of the bioactivities of the associated gene products. Preferably, such polynucleotides are those which are at least 70%, preferably at least 80% and more preferably at least 90% to 95% identical to the nucleic acid sequences at least one of: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:2 1, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, and SEQ ID NO:41.

Polynucleotides according to the invention may be employed as probes to isolate and/or identify RNA, cDNA and DNA molecules, e.g., full-length genes or polynucleotides which code for the polypeptides described herein. Likewise, the probes can be employed to isolate nucleic acids, polynucleotides or genes which have a high sequence similarity or identity with the polynucleotides of the invention.

Polynucleotides of the invention may also be used to design primers useful for the polymerase chain reaction to amplify, identify and/or isolate full-length DNA, RNA or other polynucleotides with high sequence homology or identity to the polynucleotides of the invention, as well as, polynucleotides that encode the polypeptides of the invention. Preferably, probes or primers are at least 15, 16, 17, 18, 19, 20, 21, 22, 23, 24, 25, 26, 27, 28, 29, or 30 nucleotides in length. Oligonucleotides with a length of at least 35, 40, 45, 50, 100, 150, 200, 250, or 300 nucleotides may also be used.

Methods of DNA sequencing are described inter alia by Sanger et al. (Proceedings of the National Academy of Sciences of the United States of America USA, 74:5463-5467, (1977)).

A person skilled in the art will find instructions for amplification of DNA sequences with the aid of the polymerase chain reaction (PCR) inter alia in the handbook by Gait: Oligonucleotide Synthesis: A Practical Approach (IRL Press, Oxford, UK, 1984) and in Newton and Graham: PCR 2^{nd} Edition (Springer Verlag, N.Y., 1997).

Additionally, methods employing DNA chips, microarrays or similar recombinant DNA technology that enables high throughput screening of DNA and polynucleotides that encode the herein described proteins or polynucleotides with high sequence homology or identity to the polynucleotides described herein. Such methods are known in the art and are described, for example, in Current Protocols in Molecular Biology, Ausebel et al (eds), John Wiley and Sons, Inc. New York (2000).

The polynucleotides and polypeptides of the present invention are involved in central intermediates metabolism in M. methylotrophus. By way of example, the present inventors provide the following cited references (each of which are incorporated herein by reference) demonstrating that assays to assess the enzymatic activity of the polypeptides of the present invention are known and, as such, determination of whether a sequence falls within the scope of the present claims may be readily ascertained. These polynucleotides and polypeptides include:

1. Glucose-6-phosphate isomerase enzyme comprises the amino acid sequence of SEQ ID NO:2 and is encoded by the pgi gene which comprises the polynucleotide SEQ ID NO:1 (Schreyer, R. and Bock, A., Arch. Microbiol. (1980) 127:289-298);

2. Glucose-6-phosphate 1-dehydrogenase enzyme comprises the amino acid sequence of SEQ ID NO:4 and is encoded by a zwf gene which comprises the polynucleotide SEQ ID NO:3 (Duffiieux, F. et al., J. Biol. Chem. (2000) 275: 27559-27565);

3. 6-phosphogluconolactonase enzyme comprises the amino acid sequence of SEQ ID NO:6 and is encoded by a pgl gene which comprises the polynucleotide SEQ ID NO:5 (Dufflieux, F. et al., J. Biol. Chem. (2000) 275: 27559-27565);

4. Phosphogluconate dehydratase enzyme comprises the amino acid sequence of SEQ ID NO:8 and is encoded by a edd gene which comprises the polynucleotide SEQ ID NO:7 (Egan, S. E. et. al. J. Bacteriol. (1992) 174:4638-46);

5. 2-keto-3-deoxy-6-phosphogluconate aldolase enzyme comprises the amino acid sequence of SEQ ID NO:10 and is encoded by a eda gene comprising SEQ ID NO:9 (Egan, S. E. et. al. J. Bacteriol. (1992) 174:4638-46);

6. Ribosephosphate isomerase enzyme comprises the amino acid sequence of SEQ ID NO:12 and is encoded by a rpi gene comprising SEQ ID NO:11 (Hove-Jensen, B. and Maigaard, M., J. Bacteriol. (1993) 175:5628-5635);

7. Ribulose-5-phosphate 3-epimerase enzyme comprises the amino acid sequence of SEQ ID NO:14 and is encoded by a rpe gene comprising SEQ ID NO:13 (Kiely, M. E. et. al., Biochim. Biophys. Acta (1973) 293:534-541);

8. Transketolase enzyme comprises the amino acid sequence of SEQ ID NO:16 and is encoded by a tkt gene comprising SEQ ID NO:15 (Sprenger, G. A. et. al. Eur. J. Biochem. (1995) 230:525-532);

9. Transaldolase enzyme comprises the amino acid sequence of SEQ ID NO:18 and is encoded by a tal gene comprising SEQ ID NO:17 (Sprenger, G. A. et. al. J. Bacteriol. (1995) 177:5930-5936);

10. Fructose-bisphosphatase enzyme comprises the amino acid sequence of SEQ ID NO:20 and is encoded by a fbp gene comprising SEQ ID NO:19 (Kelley-Loughnane, N. et. al., Biochim. Biophys. Acta (2002) 1594:6-16);

11. Fructose-1,6-bisphosphate aldolase enzyme comprises the amino acid sequence of SEQ ID NO:22 and is encoded by a fba gene comprising SEQ ID NO:21 (Baldwin, S. A. et. al. Biochemical. J. (1978) 169:633-641);

12. Triose phosphate isomerase 1 enzyme comprises the amino acid sequences of SEQ ID NO:24 and is encoded by a tpi1 gene comprising SEQ ID NO:23 (Anderson, A. and Cooper, R. A., FEBS Lett. (1969) 4:19-20);

13. Triose phosphate isomerase 2 enzyme comprises the amino acid sequences of SEQ ID NO:26 and is encoded by a tpi2 gene comprising SEQ ID NO:25 (Anderson, A. and Cooper, R. A., FEBS Lett. (1969) 4:19-20);

14. Triose phosphate isomerase 3 enzyme comprises the amino acid sequences of SEQ ID NO:28 and is encoded by a tpi3 gene comprising SEQ ID NO:27 (Anderson, A. and Cooper, R. A., FEBS Lett. (1969) 4:19-200);

15. Glyceraldehyde-3-phosphate dehydrogenase 1 enzyme comprises the amino acid sequences of SEQ ID NO:30 and is encoded by a gap1 gene comprising SEQ ID NO:29 (Seta, F. D. et. al., J. Bacteriol. (1997) 179:5218-5221);

16. Glyceraldehyde-3-phosphate dehydrogenase 2 enzyme comprises the amino acid sequences of SEQ ID NO:32 and is encoded by a gap2 gene comprising SEQ ID NO:31 (Seta, F. D. et. al., J. Bacteriol. (1997) 179:5218-5221);

17. Phosphoglycerate kinase enzyme comprises the amino acid sequence-of SEQ ID NO:34 and is encoded by a pgk gene comprising SEQ ID NO:33 (Bentahir, M. et. al., J. Biol. Chem. (2000) 275:11147-11153);

18. Phosphoglycerate mutase enzyme comprises the amino acid sequence of SEQ ID NO:36 and is encoded by a pgm gene comprising SEQ ID NO:35 (Fraser, H. I. et. al. FEBS Lett. (1999) 455:344-348);

19. Enolase enzyme comprises the amino acid sequence of SEQ ID NO:38 and is encoded by a eno gene comprising SEQ ID NO:37 (Spring, T. G. and Wold, F., Methods Enzymol. (1975) 42:323-329);

20. 6-phosphogluconate dehydrogenase 1 enzyme comprises the amino acid sequence of SEQ ID NO:40 and is encoded by a gnd1 gene comprising SEQ ID NO:39 (de Silva, A. O., Fraenkel, D. G., J. Biol. Chem. (1979) 254: 10237-10242);

21. 6-phosphogluconate dehydrogenase 2 enzyme comprises the amino acid sequence of SEQ ID NO:42 and is encoded by a gnd2 gene comprising SEQ ID NO:41 (de Silva, A. O., Fraenkel, D. G., J. Biol. Chem. (1979) 254: 10237-10242).

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C.

Specificity is typically the function of post-hybridization washes, the critical factors being the ionic strength and temperature of the final wash solution. For DNA—DNA hybrids, the Tm can be approximated from the equation of Meinkoth and Wahl (Anal. Biochem., 138:267-284, 1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C.

Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

Stringent hybridization conditions are understood to mean those conditions where hybridization, either in solution or on a solid support, occur between two polynucleotide molecules which are 70% to 100% homologous in nucleotide sequence which include 75%, 80%, 85%, 90%, 95%, 98% and all values and subranges therebetween.

Homology, sequence similarity or sequence identity of nucleotide or amino acid sequences may be determined conventionally by using known software or computer programs. To find the best segment of identity or similarity of sequences, BLAST (Altschul et al (1990) J. Mol. Biol. 215:403-410 and Lipman et al (1990) J. Mol. Biol. 215:403-410), FASTA (Lipman et al (1985) Science 227:1435-1441), or Smith-and Waterman (Smith and Waterman (1981) J. Mol. Biol. 147:195-197) homology search programs can be used. To perform global alignments, sequence alignment programs such as the CLUSTAL W (Thompson et al (1994) Nucleic Acids Research 22:4673-4680) can be used.

The present invention also provides processes for preparing amino acids using bacteria that comprise at least one polynucleotide whose expression is enhanced or attenuated. Likewise, the invention also provides processes for preparing amino acids using bacteria that comprise at least one polypeptide whose activity is enhanced or attenuated. Preferably, a bacterial cell with enhanced or attenuated expression of one or more of the polypeptides and/or polynucleotides described herein will improve amino acid yield at least 1% compared to a bacterial strain not having the enhanced or attenuated expression. For the production of amino acids the M. methylotrophus polynucleotides described herein may be used to target expression, either by disruption to turn off or increase or enhance the expression or relative activity of the polypeptide enzymes encoded therein.

The term “enhancement” as used herein means increasing intracellular activity of one or more polypeptides in the bacterial cell, which in turn are encoded by the corresponding polynucleotides described herein. To facilitate such an increase, the copy number of the genes corresponding to the polynucleotides described herein may be increased. Alternatively, a strong and/or inducible promoter may be used to direct the expression of the polynucleotide, the polynucleotide being expressed either as a transient expression vehicle or homologously or heterologously incorporated into the bacterial genome. In another embodiment, the promoter, regulatory region and/or the ribosome binding site upstream of the gene can be altered to achieve the over-expression. The expression may also be enhanced by increasing the relative half-life of the messenger RNA.

In another embodiment, the enzymatic activity of the polypeptide itself may be increased by employing one or more mutations in the polypeptide amino acid sequence, which increases the activity. For example, altering the relative Km of the polypeptide with its corresponding substrate will result in enhanced activity. Likewise, the relative half-life of the polypeptide may be increased.

In either scenario, that being enhanced gene expression or enhanced enzymatic activity, the enhancement may be achieved by altering the composition of the cell culture media and/or methods used for culturing.

“Enhanced expression” or “enhanced activity” as used herein means an increase of at least 10%, 25%, 50%, 75%, 100%, 150%, 200%, 300%, 400% or 500% compared to a wild-type protein, polynucleotide, gene; or the activity and/or the concentration of the protein present before the polynucleotides or polypeptides are enhanced.

The term “attenuation” as used herein means a reduction or elimination of the intracellular activity of the polypeptides in a bacterial cell that are encoded by the corresponding polynucleotide. To facilitate such a reduction or elimination, the copy number of the genes corresponding to the polynucleotides described herein may be decreased or removed. Alternatively, a weak and/or inducible promoter may used to direct the expression of the polynucleotide, the polynucleotide being expressed either as a transient expression vehicle or homologously or heterologously incorporated into the bacterial genome. For example, the endogenous promoter or regulatory region of the gene corresponding to the isolated polynucleotides described herein may be replaced with the aforementioned weak and/or inducible promoter. Alternatively, the promoter or regulatory region may be removed. The expression may also be attenuated by decreasing the relative half-life of the messenger RNA.

In another embodiment, the enzymatic activity of the polypeptide itself may be decreased or deleted by employing one or more mutations in the polypeptide amino acid sequence, which decreases the activity or removes any detectable activity. For example, altering the relative Kd of the polypeptide with its corresponding substrate will result in attenuated activity. Likewise, a decrease in the relative half-life of the polypeptide will result in attenuated activity.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

Suitable vectors for carrying M. methylotrophus polynucleotides include those vectors which can direct expression of the gene in bacterial cells as known in the art. One embodiment of the present invention is whereby the vectors contain an inducible or otherwise regulated expression system whereby the M. methylotrophus polynucleotides may be expressed under certain conditions and not expressed under other conditions. Furthermore, in another embodiment of the invention, the M. methylotrophus polynucleotides can be constitutively expressed. Examples of such vectors and suitable cells in which they can be introduced are described in Sambrook et al., Molecular Cloning: A Laboratory Manual, 3rd Ed., Cold Spring Harbor Laboratory Press, Cold Spring Harbor, N.Y. (2001) and Current Protocols in Molecular Biology, Ausebel et al, (Eds.), John Wiley and Sons, Inc., New York, 2000.

Methods of introducing M. methylotrophus polynucleotides or vectors containing the M. methylotrophus polynucleotides include electroporation, conjugation, calcium-mediated transfection, infection with bacteriophage and other methods known in the art. These and other methods are described in Sambrook et al., Molecular Cloning: A Laboratory Manual, 3rd Ed., Cold Spring Harbor Laboratory Press, Cold Spring Harbor, N.Y. (2001) and Current Protocols in Molecular Biology, Ausebel et al, (Eds.), John Wiley and Sons, Inc., New York (2000). M. methylotrophus which have the ability to produce amino acids are described in EP 1188822 A1. The ability to produce amino acids can be imparted to M. methylotrophus by mutagenesis of a gene controlling the amino acid biosynthesis, or by enhancing the activity of one or more enzymes involved in the amino acid biosynthesis. Introduction of a gene involved in excretion of amino acids is also effective for improving amino acid productivity. Examples of M. methylotrophus having the ability to produce L-lysine include strains into which a mutant lysE gene and a mutant dapA gene have been introduced. A mutant lysE gene is a homologue of the lysE gene isolated from Corynebacterium glutamicum and promotes excretion of L-lysine when it is introduced into a methylotroph (U.S. 2003/0124587 A1, US 2004/0146974 A1). A mutant dapA gene encodes dihydrodipicolinate synthase which is not subject to feedback inhibition by L-lysine (U.S. 2003/0124587 A1). M. methylophilus AS1/pRSlysEdapA is exemplified (U.S. 2003/0124587 A1).

The microorganisms that can be used in the present invention should have the ability to produce amino acids, preferably L-amino acids, from a suitable carbon source, preferably carbon sources such as methanol, glucose, sucrose, lactose, fructose, maltose, molasses, starch, cellulose glycerol or ethanol. The microorganisms can be Methylophilus bacteria, preferably Methylophilus methylotrophus.

Suitable culture conditions for the growth and/or production of M. methylotrophus polynucleotides are dependent on the cell type used. Likewise, culturing cells that contain attenuated or enhanced expression of the M. methylotrophus polynucleotides or polypeptides, as described herein, may be cultured in accordance with methods known in the art. Examples of culture conditions for various cells is described in Sambrook et al., Molecular Cloning: A Laboratory Manual, 3rd Ed., Cold Spring Harbor Laboratory Press, Cold Spring Harbor, N.Y. (2001); Current Protocols in Molecular Biology, Ausebel et al, (Eds.), John Wiley and Sons, Inc., 2000; and Cells: A Laboratory Manual (Vols. 1-3), Spector et al, (Eds.), Cold Spring Harbor Laboratory Press, Cold Spring Harbor, N.Y., (1988).

Following culturing the polypeptide or protein products, which are encoded by the M. methylotrophus polynucleotides, may be purified using known methods of protein purification. These methods include high performance liquid chromatography (HPLC), ion-exchange chromatography, size exclusion chromatography; affinity separations using materials such as beads with exposed heparin, metals, or lipids; or other approaches known to those skilled in the art. These and other methods of protein purification are disclosed in Sambrook et al., Molecular Cloning: A Laboratory Manual, 3rd Ed., Cold Spring Harbor Laboratory Press, Cold Spring Harbor, N.Y. (2001); Current Protocols in Molecular Biology, Ausebel et al, eds., John Wiley and Sons, Inc., 2000 and Protein Purification, Scopes and Cantor, (Eds.), Springer-Verlag, (1994). Likewise, the amino acids produced may be purified by methods known in the art using similar chromatography devices.

The invention also provides antibodies that bind to the polypeptides of the present invention. Antibodies binding to the polypeptides can be either monoclonal or polyclonal, preferably the antibodies are monoclonal. Methods for obtaining antibodies that bind to the polypeptides are known in the art and are described, for example, in Harlow and Lane, Antibodies: A Laboratory Manual, Cold Spring Harbor Laboratory Press, Cold Spring Harbor, N.Y. (1988).

Having generally described this invention, a further understanding can be obtained by reference to certain specific examples which are provided herein for purposes of illustration only, and are not intended to be limiting unless otherwise specified.

### EXAMPLES

Whole genome sequencing using random shotgun method is described in Fleischman R. D. et. al. (1995) Science, 269: 496-512.

### Example 1

### Construction of Genomic Libraries of Methylophilus Methylotrophus

M. methylotrophus AS1 was cultured at 30° C. in the 121 medium described in the Catalogue of Strains (The National Collections of Industrial and Marine Bacteria Ltd., 1994).

Cells were collected by centrifugation. Genomic DNA was isolated using the Genome-tip system (Qiagen K. K., Tokyo, Japan). The genomic DNA was sheared and fragmentized by sonication. The resultant fragments in the 1- to 2-kb size range were purified by gel electrophoresis through 1% low-melting agarose gel, followed by recovery using the Wizard DNA purification kit (Promega KK, Tokyo, Japan). The recovered fragments were ligated to the high-copy number vector pUC 118 treated by Hincil and bacterial alkaline phosphatase (Takara Shuzo, Kyoto, Japan), and this was designated pUC118 library.

For larger fragments (9- to 11-kb in size), the genomic DNA was partially digested by restriction endonuclease Sau3AI, followed by 0.6% agarose gel electrophoresis. The DNA fragments corresponding 9-kb to 11-kb in size were excised from gel and were recovered using the DNACELL (Daiichi Pure Chemicals, Tokyo, Japan). The recovered fragments were ligated into the low-coy number vector pMW118 (Nippon Gene, Toyama, Japan), which is a derivative of the pSC101 (Bernaidi, A. and Bernardi, F. (1984) Nucleic Acids Res. 12, 9415-9426). This library composed of large DNA fragments was designated pMW118 library.

General DNA manipulation was performed according to previously described methods (Sambrook et. al. (1989) “Molecular Cloning: A Laboratory Manual/Second Edition”, Cold Spring Harbor Laboratory Press).

### Example 2

### DNA Sequencing and Sequence Assembly

The pUC118 library were transformed into Escherichia coli DH5α and plated on Luria-Bertani medium containing 100 μg/ml ampicillin and 40 μg/ml 5-bromo-4-chloro-3-indolyl-α-D-galactoside (X-Gal). The white colonies were picked up and cultured in Luria-Bertani medium containing 100 μg/ml ampicillin. The individual colony was cultured in the well of the 96 deep-well plates, and the plasmids were isolated using QIAprep Turbo Kit (Qiagen). The DNA fragments inserted into pUC118 were sequenced using a Ml 3 reverse primer. The shotgun sequencing was performed with the BigDye terminators and 3700 DNA analyzer (Applied Biosystems Japan, Tokyo, Japan). Approximately 50,000 samples from pUC118 library corresponding to coverage of approximately 8-fold to the genome size were analyzed and the sequences were assembled by Phred/Phrap software (CodonCode, Mass., USA). This assembly treatment yielded 60 contigs with more than 5 kb in size.

As for pMW118 library, 2,000 clones corresponding to coverage of approximately 5-fold were sequenced using both M13 forward and reverse primers. The end-sequence data were analyzed and the linking clones between contigs were selected from pMW118 library. The inserted fragments of selected clones were amplified by the polymerase chain reaction (PCR) using LA Taq polymerase (Takara Shuzo) and M. methylotrophus genomic DNA as a template. These products of PCR were entirely sequenced as described in Example 1, and the gap DNA sequences between contigs were determined. By the additional sequence information, the Phrap assembly software reduced the number of contigs with more than 5 kb in size to 24. Then the 48 DNA primers with sequences complementary to the end-sequences of the 24 contigs were prepared. All possible pairwise combination of the primers were tested by PCR to amplify the DNA fragments of M. methylotrophus genomic DNA. The amplified products were sequenced directly. In several cases, the additional primers complementary to different sequences at the end of the contig were used. This strategy could close all of the remaining physical gaps and resulted in a single circular contig. Several regions that had been sequenced in only one direction and had postulated secondary structure were confirmed. By this research, the genome of M. methylotrophus was found to be a single circular with the size of 2,869,603 bases and GC content of 49.6%.

### Example 3

### Sequence Analysis and Annotation

Sequence analysis and annotation was managed using the Genome Gambler software (Sakiyama, T. et. al. (2000) Biosci. Biotechnol. Biochem. 64: 670-673). All open reading frames of more than 150 bp in length were extracted and the translated amino acid sequences were searched against non-redundant protein sequences in GenBank using the BLAST program (Altschul, S. F. et. al. (1990) J. Mol. Biol. 215, 403-410). Of putative polynucleotide encoding sequences with significant similarities to the sequences in public databases (BLASTP scores of more than 100), the genes involved in biosynthesis of amino acids were selected. Start codons (AUG or GUG) were putatively identified by similarity of the genes and their proximity to the ribosome binding sequences (Shine, J. and Dalgarno, L. (1975) Eur. J. Biochem. 57: 221-230). Careful assignment of gene function resulted in the identification of the glucose-6-phosphate isomerase gene (pgi), the glucose-6-phosphate 1-dehydrogenase gene (zwf), the 6-phosphogluconolactonase gene (pgl), the 6-phosphogluconate dehydrogenase genes (gnd1 and gnd2), the fructose-bisphosphatase gene (fbp), and the fructose-1,6-bisphosphate aldolase gene (fba). The two enzymes of the Entner-Doudoroff pathway, phosphogluconate dehydratase gene (edd) and 2-keto-3-deoxy-6-phosphogluconate aldolase gene (eda) were found probably in operon. The ribosephosphate isomerase gene (rpi), ribulose-5-phosphate 3-epimerase gene (rpe), transketolase gene (tkt), and the transaldolase gene (tal) in the reversible reaction on Pentose-Phosphate cycles were identified. The glyceraldehyde-3-phosphate dehydrogenase genes (gap1 and gap2), phosphoglycerate kinase gene (pgk) the phosphoglycerate mutase (pgm), the enolase gene (eno), and the triose phosphate isomerase genes (tpi1, tpi2, and tpi3) were also identified.

The reagents used in the following examples were obtained from Wako Pure Chemicals or Nakarai Tesque unless otherwise indicated. The compositions of the media used in each example are shown below. pH was adjusted with NaOH or HCl for all of the media.

LB Medium:

| | | |
|---|---|---|
| Trypton peptone (Difco) | 10 | g/L |
| Yeast extract (Difco) | 5 | g/L |
| NaCl | 10 | g/L |

| | |
|---|---|
| pH | 7.0 |

These were steam-sterilized at 120° C. for 20 minutes.

LB Agar Medium:

LB medium

| | |
|---|---|
| Bacto agar | 15 g/L |

These were steam-sterilized at 120° C. for 20 minutes.

SEII Medium:

(Refer to Journal of General Microbiology(1989) 135, 3153-3164, Silman N. J., Carver M. A. & Jones C. W. Some minor adjustments are made.)

| | | |
|---|---|---|
| K₂HPO₄ | 1.9 | g/L |
| NaH₂PO₄ | 1.56 | g/L |
| MgSO₄.7H₂O | 0.2 | g/L |
| (NH₄)₂SO₄ | 5 | g/L |
| CuSO₄.5H₂O | 5 | μg/L |
| MnSO₄.4-5H₂O | 25 | μg/L |
| ZnSO₄.7H₂O | 23 | μg/L |
| CaCl₂.2H₂O | 72 | mg/L |
| FeCl₃.6H₂O | 9.7 | mg/L |
| CaCO₃ (Kanto Kagaku) | 30 | g/L |
| Methanol | 2% | (vol/vol) |

| | |
|---|---|
| pH | 7.0 |

Except for methanol, the components were steam-sterilized at 121° C. for 15 minutes. After the components were sufficiently cooled, methanol was added.

SEII Agar Medium:

| K₂HPO₄ | 1.9 | g/L |
|---|---|---|
| NaH₂PO₄ | 1.56 | g/L |
| MgSO₄.7H₂O | 0.2 | g/L |
| (NH₄)₂SO₄ | 5 | g/L |
| CuSO₄.5H₂O | 5 | μg/L |
| MnSO₄.4-5H₂O | 25 | μg/L |
| ZnSO₄.7H₂O | 23 | μg/L |
| CaCl₂.2H₂O | 72 | mg/L |
| FeCl₃.6H₂O | 9.7 | mg/L |
| CaCO₃ (Kanto Kagaku) | 30 | g/L |
| Methanol | 2% | (vol/vol) |
| pH | 7.0 | |
| Bacto agar (Difco) | 15 | g/L |

Except for methanol, the components were steam-sterilized at 121° C. for 15 minutes. After the components were sufficiently cooled, methanol was added.

### Example 4

### The Effect of Amplification of the Tal Gene in Methylophilus Bacterium

The introduction of an L-lysine biosynthetic enzyme gene (dapA*) and a gene (lysE24) having L-lysine excretion activity into a Methylophilus bacterium causes lysine to accumulate in the medium. dapA* encodes a dihydrodipicolinate synthase that is free from L-lysine feedback inhibition. Furthermore, lysE24 is a mutant form of the lysE gene that exhibits L-lysine excretion activity in methanol-utilizing bacteria. Accordingly, these genes were introduced to examine the effect on amplification of the tal gene in a Methylophilus bacterium which causes accumulation of L-lysine.

<1> Introduction of an L-lysine biosynthetic enzyme gene (dapA*) and a gene (lysE24) having L-lysine excretion activity on Methylophilus bacteria.

(1) Construction of pBGEA

To introduce the dapA* and LysE24 genes into a Methylophilus bacterium, the known plasmid pBHRI (Antoine, R. and Locht, C., Mol. Microbiol., 6, 1785-99 (1992)) was employed to construct a dapA* and LysE24-expression plasmid, pBGEA. First, pBHRI was digested with the restriction enzyme Dra1, after which a phenol chloroform solution was admixed to stop the reaction. The reaction mixture was separated in a centrifuge. The supernatant was recovered and precipitated from ethanol, yielding DNA. The ends of the recovered DNA fragments were blunted using a DNA blunting kit (TAKARA BIO INC.).

The dapA* and LysE24 genes were obtained from the plasmid pRSlysEdapA (see US 2003/0124687 A1), which contains these genes. The E. coli JM109 strain transformed with the pRSlysEdapA plasmid was designated as AJ13832, and this strain was deposited at the independent administrative corporation, National Institute of Advanced Industrial Science and Technology, International Patent Organism Depositary on Jun. 4, 2001 and received an accession number of FERM P-18371. Then, it was converted to an international deposit under the provisions of the Budapest Treaty on May 13, 2002, 2002, and received an accession number of FERM BP-8042.

First, the pRSlysEdapA was digested with the restriction enzymes EcoRI and BgIII, after which a phenol chloroform solution was admixed to stop the reaction. The reaction mixture was separated in a centrifuge. The supernatant was recovered and precipitated from ethanol, yielding DNA. Subsequently, 0.8 percent agarose gel electrophoresis was employed to separate the targeted DNA fragments, and DNA fragments of about 2.0 Kbp were recovered with an EASY TRAP ver. 2 (DNA collection kit, TAKARA BIO INC.). The recovered ends of the DNA fragments were blunted and phosphorylated with a BKL kit (TAKARA BIO INC.).

The pBHRI digestion product prepared as set forth above and the fragment comprising the dapA* and LysE24 genetic regions were ligated using a DNA Ligation Kit, Ver. 2 (TAKARA BIO INC.). Escherichia coli (E. coli JM109 competent cells, TAKARA BIO INC.) was transformed with the ligation reaction mixture, plated on LB agar medium containing 20 mg/L of kanamycin, and maintained at a temperature of 37° C. overnight. The colonies that appeared on the agar medium were inoculated onto LB liquid medium containing 20 mg/L of kanamycin and cultured for eight hours at 37° C. with shaking. Plasmid DNA was extracted from the various culture solutions by alkali SDS. The structure was confirmed by digestion with restriction enzymes and base pair sequencing. A plasmid in which the transcription directions of the chloramphenicol resistance gene, dapA* gene, and lysE24 gene were identical was selected as pBHR-EA.

A plasmid pBGEA into which had been incorporated a gentamycin-resistance marker was constructed from the pBHR-EA thus obtained.

First, the pBHR-EA was digested with the restriction enzyme Ncol and a phenol chloroform solution was admixed to stop the reaction. The reaction product was separated by centrifugation. The supernatant was collected and precipitated from ethanol to obtain DNA. The ends of the DNA fragments collected were blunted using a DNA Blunting Kit (TAKARA BIO INC.).

Furthermore, the gentamycin-resistance gene region was amplified employing a known plasmid pML122 (Monika Labes, Alfred Puhler, and Reinhard Simon, Gene, 89, (1990), 37-46) as template DNA and employing the DNA primers represented by SEQ ID NOS: 43 and 44 by PCR (denaturation for 10 s at 94° C., annealing for 30 s at 60° C., and an elongation reaction for 90 s at 72° C.). Pyrobest DNA polymerase (TAKARA BIO INC.) was employed in the PCR. The ends of the gentamycin-resistance gene region fragment obtained were blunted and phosphorylated with a BKL Kit (TAKARA BIO INC.). The pBHR-EA digestion product and gentamycin-resistance gene region fragments thus prepared were ligated using a DNA Ligation Kit Ver. 2 (TAKARA BIO INC.). Escherichia coli (E. coli JM109 competent cells, TAKARA BIO INC.) was transformed with the ligation reaction mixture, plated on LB agar medium comprising 50 mg/L of gentamycin, and maintained at 37° C. overnight. The colonies appearing on the agar medium were inoculated onto an LB liquid medium containing 50 mg/L of gentamycin and cultured for 8 hours at 37° C. with shaking. Plasmid DNA was extracted from the individual culture solutions by alkali SDS and the structure was confirmed by digestion with restriction enzymes and base pair sequencing, yielding pBGEA.

(2) Introduction of pBGEA into Methylophilus Bacteria

The pBGEA obtained as set forth above was introduced into Methylophilus methylotrophus AS1 strain (NCIMB 10515) by electroporation (Canadian Journal of Microbiology, 43, 197 (1997)). Transformants (referred to as “AS1/pBGEA” hereinafter) were selected using gentamycin tolerance as an indicator.

<2> Amplification of the tal gene in Methylophilus Bacteria Accumulating L-lysine

Construction of pRStal

To introduce the tal gene into Methylophilus bacteria, the known plasmid pRS was employed to construct a plasmid pRStal for expression of tal. pRS is a plasmid having only the vector segment, obtained by eliminating the DNA region encoding the threonine operon, of the pVIC40 plasmid (US 5175107), the latter being derived from the broad host spectrum vector plasmid pAYC32 (Chistorerdov, A. Y., Tsygankov, Y. D., Plasmid, 1986, 16, 161-167), a derivative of RSF1010.

First, pRS was digested with the restriction enzyme EcoRI. A phenol chloroform solution was admixed to stop the reaction. The reaction product was separated by centrifugation, the supernatant was collected, precipitation from ethanol was conducted, and DNA was recovered. The ends of the DNA fragments that were recovered were blunted with a DNA Blunting Kit (TAKARA BIO INC.).

The tal gene was amplified by PCR (denaturation for 10 s at 94° C., annealing for 30 s at 60° C., and an elongation reaction for 120 s at 72° C.) employing the DNA primers shown in SEQ ID NOS: 45 and 46 and employing chromosomes extracted from Methylophilus methylotrophus as template. Pyrobest DNA polymerase (TAKARA BIO INC.) was employed in the PCR. The ends of the tal gene fragments obtained were blunted and phosphorylated with a BKL kit (TAKARA BIO INC.).

The pRS digestion production and tal gene region fragments thus prepared were ligated with a DNA Ligation Kit Ver. 2 (TAKARA BIO INC.). Escherichia coli (E. coli JM109 competent cells, TAKARA BIO INC.) was transformed with the ligation reaction mixture, plated on LB agar medium comprising 20 mg/L of streptomycin, and maintained at 37° C. overnight. The colonies appearing on the agar medium were inoculated onto an LB liquid medium containing 20 mg/L of streptomycin and cultured for 8 hours at 37° C. with shaking. Plasmid DNA was extracted from the individual culture solutions by the alkali SDS method and the structure was confirmed by digestion with restriction enzymes and base pair sequencing, yielding pRStal.

(2) The Introduction of pRStal into AS1/pBGEA and the Production of Amino Acid

The pRStal obtained as set forth above was introduced into AS I/pBGEA by electroporation (Canadian Journal of Microbiology, 43, 197 (1997)). The transformants obtained (referred to hereinafter as “AS1/pBGEA/pRStal”) and a control in the form of a strain into which pRS had been introduced (referred to as “AS1/pGBEA/pRS” hereinafter) were cultured and the L-lysine concentration of the supernatants was measured in the following manner.

Each transformant was broadly coated on SEH agar medium containing 50 mg/L of gentamycin and 20 mg/L of streptomycin and cultured overnight at 37° C. Next, roughly 10 cm² of the bacterial mass on the surface of the medium was scraped off, transplanted to SEII production medium (20 mL) containing 50 mg/L of gentamycin and 20 mg/L of streptomycin, and cultured for 13 h at 37° C. with shaking. Following completion of culturing, the bacterial mass was removed by centrifugal separation and the L-lysine concentration in the culture supernatant was measured with a Biotech Analyzer AS210 (Sakura Seiki). The results are given in Table 1. The tal gene amplification strain AS1/pBGEA/pRStal exhibited greater accumulation of L-lysine in the medium than the control AS1/pGBEA/pRS strain.

TABLE 1

| Bacterial Strain | L-Lysine Production (mg/L) |
|---|---|
| AS1/pGBEA/pRS | 290 |
| AS1/pBGEA/pRStal | 390 |

### Example 5

### Effect of Amplification of rpi Gene in Methylophilus Bacterium

The effects of the amplification of the rpi gene were examined in a Methylophilus bacterium which causes accumulation of L-lysine by introducing the dapA* and lysE24 genes.

<1> Amplification of the rpi Gene in Methylophilus Bacterium Which Causes Accumulation of L-lysine

(1) Construction of pAYCTER3 Plasmid

The synthetic DNA described in SEQ ID NOS: 47 and 48, designed to contain the sequence of the multicloning site of pUC19 (TAKARA BIO INC.), was annealed by methods well-known to those of ordinary skill in the art to prepare a polylinker. This polylinker was designed to have the same end shapes as those achieved by cleaving with restriction enzymes EcoRI and Bg1II. Furthermore, the primers of SEQ ID NOS: 49 and 50 were synthesized as primers, and the region coding for the terminator sequence of rrnB was amplified by PCR with chromosomal DNA of Escherichia coli K-12 prepared by the usual method (the method of Saito and Miura (Biochim. Biophys. Acta, 72, 619 (1963))) as template. A sequence recognized by the restriction enzyme Bg1II was designed into the primer of SEQ ID NO:47, and a sequence recognized by restriction enzyme BcII was designed into the primer of SEQ ID NO:48. PCR was conducted with Pyrobest DNA polymerase (TAKARA BIO INC.) according to the protocol recommended by those in the trade. After digesting the PCR fragments with the restriction enzymes Bg1II and Bc1I, the PCR fragments and the above polylinker were ligated to prepare DNA fragments of about 400 bp. A DNA Ligation Kit Ver. 2.1 (TAKARA BIO INC.) was employed in the ligation reaction; reaction conditions were in accordance with the recommended protocol of those in the trade. Fragments of about 9.2 kbp that had been cut out of the known plasmid pAYC32 (J. Gen. Microbiol., 137, 169-178 (1991)) with the restriction enzymes EcoRI and BamHI were recovered and the above DNA fragments were inserted to construct an expression plasmid pAYCTER3 that functioned in Methylophilus methylotrophus AS1.

In this structure, pAYCTER3 lacked the 5′ side upstream sequence of the strA gene encoded in pAYC32, having instead a pUC19 multicloning site and an rrnB terminator.

(2) Construction of pAYCTER3-rpi

To introduce the rpi gene into a Methylophilus bacterium, the pAYCTER3 construction in (1) above was employed and the plasmid pAYCTER3-rpi for rpi expression was constructed.

First, pSTV28 (TAKARA BIO INC.) was digested with the restriction enzyme XbaI and precipitated from ethanol to purify the DNA. Additionally, the rpi gene was amplified by PCR (denaturation for 10 s at 94° C., annealing for 30 s at 57° C., and elongation for 60 s at 68° C.) using the DNA primers denoted by SEQ ID NOS: 51 and 52 with chromosomes extracted from Methylophilus methylotrophus AS1 as a template. Pyrobest DNA polymerase (TAKARA BIO INC.) was employed in PCR. The PCR product was digested with the restriction enzyme Xbal and precipitated from ethanol to purify the DNA.

The pSTV28 digestion product and rpi gene region fragments prepared as set forth above were ligated using a DNA ligation Kit Ver. 2 (TAKARA BIO INC.). Escherichia coli (E. coli JM109 competent cells, TAKARA BIO INC.) was transformed with the ligation reaction mixture, plated on LB agar medium containing 25 mg/L of chloramphenicol, 40 mg/L of X-gal, and 100 μM of PTG, and maintained at 37° C. overnight. The white colonies that appeared on the agar medium were inoculated onto LB liquid medium containing 25 mg/L of chloramphenicol and cultured for 8 hours at 37° C. with shaking. The plasmid DNA was extracted from the culture solutions by alkali SDS and the structure was confirmed by digestion with restriction enzymes and base sequencing, yielding pSTV28-rpi.

Next, a DNA fragment obtained by digesting pSTV28-rpi with XbaI and purifying with precipitation in ethanol and a DNA fragment obtained by digesting with XbaI the pAYCTER3 constructed in (2) followed by precipitation from ethanol were ligated with a DNA Ligation Kit Ver.2 (TAKARA BIO INC.). Escherichia coli (E. coli JM109 competent cells, TAKARA BIO INC.) was transformed with the ligation reaction mixture, plated on LB agar medium containing 100 mg/L of ampicillin and 20 mg/L of streptomycin, and maintained at 37° C. overnight. The colonies that appeared on the agar medium were inoculated onto LB liquid medium containing 100 mg/L of ampicillin and 20 mg/L of streptomycin and cultured for 8 hours at 37° C. with shaking. The plasmid DNA was extracted from the culture solutions by alkali SDS and the structure was confirmed by digestion with restriction enzymes and base sequencing, yielding pAYCTER3-rpi.

<2> Introducon of pAYCTER3-rpi into AS1/pBGEA and the Production of Amino Acid

The pAYCTER3-rpi obtained as set forth above was introduced into AS1/pBGEA by electroporation (Canadian Journal of Microbiology, 43, 197 (1997)). The transformants obtained (referred to hereinafter as “AS1/pBGEA/pAYCTER3-rpi”) and a control in the form of AS1/pGBEA were cultured and the L-lysine concentration of the culture supernatant was measured in the following manner.

The transformants were broadly coated on SEII agar medium containing 50 mg/L of gentamycin and 20 mg/L of streptomycin and cultured overnight at 37° C. Next, roughly 10 cm² of the bacterial mass on the surface of the medium was scraped off, transplanted to SEII production medium (20 mL) containing 50 mg/L of gentamycin and 20 mg/L of streptomycin, and cultured for 24 h at 37° C. with shaking. The control strain AS1/pBGEA was similarly cultured on SEII medium containing 50 mg/L of gentamycin. Following completion of culturing, the bacterial mass was removed by centrifugal separation and the L-lysine concentration in the culture supernatant was measured with a Biotech Analyzer AS210 (Sakura Seiki). The results are given in Table 2. The rpi gene amplification strain AS1/pBGEA/pAYCTER3-rpi exhibited greater accumulation of L-lysine in the medium than the control AS1/pGBEA strain.

TABLE 2

| Bacterial Strain | L-Lysine Production (mg/L) |
|---|---|
| AS1/pGBEA | 680 |
| AS1/pBGEA/pAYCTER3-rpi | 708 |

### Example 6

### Effect of Combined Amplification of tal Gene and rpi Gene in Methylophilus Bacterium

The combined amplification effect of the tal gene and the rpi gene on a Methylophilus bacterium which causes accumulation of L-lysine by introduction of the dapA* and lysE24 genes was examined.

<1> Amplification of tal Gene and rpi Gene in Methylophilus Bacterium Which Causes Accumulation of L-Lysine

Construction of pRS−tal+rpi

To introduce the tal gene and rpi gene into a Methylophilus bacterium, an expression plasmid pRS−tal+rpi carrying the rpi gene in the pRStal plasmid constructed in <2> of Example 4 was constructed. First, pRStal was digested with the restriction enzyme Xbal, subjected to end blunting with a DNA Blunting Kit (TAKARA BIO INC.), and precipitated from ethanol to purify the DNA.

Additionally, for the rpi gene, the pAYCTER3-rpi constructed in <1> (2) in Example 5 was digested with XbaI, subjected to end blunting, and precipitated from ethanol to purify the DNA.

The pRStal digestion product and rpi gene region fragment prepared in this manner were ligated with a DNA Ligation Kit Ver.2 (TAKARA BIO INC.). Escherichia coli (E. coli JM109 competent cells, TAKARA BIO INC.) was transformed with the ligation reaction mixture, plated on LB agar medium containing 20 mg/L of streptomycin, and maintained at 37° C. overnight. The colonies that appeared on the agar medium were inoculated onto LB liquid medium containing 20 mg/L of streptomycin and cultured for 8 hours at 37° C. with shaking. The plasmid DNA was extracted from the culture solutions by alkali SDS and the structure was confirmed by digestion with restriction enzymes and base sequencing, yielding pRS−tal+rpi.

<2> The Introduction of pRS−tal+rpi into AS1/pBGEA and the Production of Amino Acid

The pRS−tal+rpi obtained as set forth above was introduced into As1/pBGEA by electroporation (Canadian Journal of Microbiology, 43, 197 (1997)). The transformants obtained (referred to hereinafter as “AS1/pBGEA/pRS−tal+rpi”) and a control strain in the form of AS1/pBGEA were cultured and the L-lysine concentration of the culture supernatants was measured in the following manner.

Each transformant was broadly coated on SEII agar medium containing 50 mg/L of gentamycin and 20 mg/L of streptomycin and cultured overnight at 37° C. Next, roughly 10 cm² of the bacterial mass on the surface of the medium was scraped off, transplanted to SEII production medium (20 mL) containing 50 mg/L of gentamycin and 20 mg/L of streptomycin, and cultured for 24 h at 37° C. with shaking. The control strain AS1/pBGEA was similarly cultured in SEII medium containing 50 mg/L of gentamycin. Following completion of culturing, the bacterial mass was removed by centrifugal separation and the L-lysine concentration in the culture supernatant was measured with a Biotech Analyzer AS2 10 (Sakura Seiki). The results are given in Table 3. The tal gene and rpi gene combined amplification strain AS1/pBGEA/pRStal+rpi exhibited greater accumulation of L-lysine in the medium than the control AS1/pGBEA strain.

TABLE 3

| | Level of L-Lysine |
|---|---|
| Bacterial Strain | Production (mg/L) |
| AS1/pGBEA | 680 |
| AS1/pBGEA/pRS-tal + rpi | 828 |

Obviously, numerous modifications and variations on the present invention are possible in light of the above teachings. It is therefore to be understood that within the scope of the appended claims, the invention may be practiced otherwise than as specifically described herein.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. An isolated polynucleotide encoding a protein comprising the amino acid sequence selected from the group consisting of SEQ ID NO:12, SEQ ID NO:18, and combinations thereof.

2. A vector comprising at least one polynucleotide of claim 1.

3. A host cell comprising at least one polynucleotide of claim 1.

4. The host cell of claim 3 comprising a Methylophilus bacterium.

5. The host cell of claim 4 comprising a Methylophilus methylotrophus bacterium.

6. A method of producing at least one amino acid comprising
culturing the host cell of claim 3 for a time and under conditions suitable for producing the amino acid; and
collecting the amino acid produced.

7. The method of claim 6, wherein said at least one amino acid comprises an L-amino acid.

8. The method of claim 6, wherein said at least one amino acid comprises L-lysine.

9. The method of claim 6, wherein said host cell comprises a Methylophilus bacterium.

10. The method of claim 6, wherein said host cell comprises a Methylophilus methylotrophus bacterium.

11. An isolated polynucleotide comprising a nucleotide sequence selected from the group consisting of SEQ ID NO:11, SEQ ID NO:17, and combinations thereof.

12. A vector comprising at least one polynucleotide of claim 11.

13. A host cell comprising at least one polynucleotide of claim 11.

14. The host cell of claim 13 comprising a Methylophilus bacterium.

15. The host cell of claim 13 comprising a Methylophilus methylotrophus bacterium.

16. A method of producing at least one amino acid comprising
culturing the host cell of claim 13 for a time and under conditions suitable for producing the amino acid; and
collecting the amino acid produced.

17. The method of claim 16, wherein said at least one amino acid comprises an L-amino acid.

18. The method of claim 16, wherein said at least one amino acid comprises L-lysine.

19. The method of claim 16, wherein said host cell comprises a Methylophilus bacterium.

20. The method of claim 16, wherein said host cell comprises a Methylophilus methylotrophus bacterium.

21. An isolated polynucleotide which hybridizes under stringent conditions comprising hybridization in 50% formamide, 1M NaCl, 1% SDS at 37° C. followed by washing in 0.1× SSC at 60° C. to 65° C., to at least one of the isolated polynucleotides of claim 11.

22. A vector comprising the isolated polynucleotide of claim 21.

23. A host cell comprising the isolated polynucleotide of claim 21.

24. The host cell of claim 23, wherein said host cell comprises a Methylophilus bacterium.

25. The host cell of claim 23, wherein said host cell comprises a Methylophilus methylotrophus bacterium.

26. A method of producing at least one amino acid comprising
culturing the host cell of claim 23 for a time and under conditions suitable for producing the amino acid; and
collecting the amino acid produced.

27. The method of claim 26, wherein said at least one amino acid comprises an L-amino acid.

28. The method of claim 26, wherein said at least one amino acid comprises L-lysine.

29. The method of claim 26, wherein said host cell comprises a Methylophilus bacterium.

30. The method of claim 26, wherein said host cell comprises a Methylophilus methylotrophus bacterium.

31. An isolated polynucleotide which is at least 95% identical to the polynucleotide of claim 11.

32. A vector comprising the isolated polynucleotide of claim 31.

33. A host cell comprising the isolated polynucleotide of claim 31.

34. The host cell of claim 33, wherein said host cell comprises a Methylophilus bacterium.

35. The host cell of claim 33, wherein said host cell comprises a Methylophilus methylotrophus bacterium.

36. A method of producing at least one amino acid comprising
culturing the host cell of claim 33 for a time and under conditions suitable for producing the amino acid; and
collecting the amino acid produced.

37. The method of claim 36, wherein said at least one amino acid comprises an L-amino acid.

38. The method of claim 36, wherein said amino acid comprise L-lysine.

39. The method of claim 36, wherein said host cell comprises a Methylophilus bacterium.

40. The method of claim 36, wherein said host cell comprises a Methylophilus methylotrophus bacterium.


SEQUENCE LISTING (52 sequences)

SEQ ID NO: 1 - DNA, 1653 bp, Methylophilus methylotrophus
  CDS (1)..(1653)
atggcaaaattaaccacacacccgagctggcacgcgctgaaccagcatcaaattgaaatgcgcaatgttcaaatgcgcgacctgttcaaacaaaacccgaaacgatttgaagagttcagcctcaccgtagaagatgtcctgcttgactactccaaacaccgcattacaaaagaaaccttagcacacctgtttcaactggcgcgtgactccaatattgagcactggcgcagccgcatgttcagcggtgaaaaaatcaatatcaccgaaaatcgcgccgtactgcataccgcactacgcaaccgtagcaacacacctgtgtatgtcgatggcaaggatgtgatgccagaagtgaatgcggttctcgcacaaatgcgtagctttactgagcaggtgcgtagcggtcagtggactggttacactggcaagcgaattaccgatatcgtgaatattggtatcggcggctccgatttggggcctgtcatggcttgcgatgccttgaaaccttacgccagtccggatttaaaagcgcattttgtttccaacattgatggtgcgcacctgatgcgtgtactggaaacctgtcatccggaaactaccctgtttattgtggcgtcgaaaacattcaccacgcaggaaaccatgaccaacgcccactccgcgcgccgctggttcctaaacgcagcccagcaagaccaacatgtggccaaacattttgtggctttatccaccaatgccaaagcggtacaggcattcggcatagataccaacaacatgtttgctttctgggactgggtgggtggccgttactccttgtggtcagccatcggtttatcgatcgcattgtatgtgggcatggacaactttgaacaaatgttggctggtggacacgccatggaccagcacttccagactgcgccgttggaacagaacatgcctgttatcatggcattgctcggcatctggtataacaacttcttccatgtggatactcacgcgatcttgccttacgaccaggggatgtctcgttttgcagcttatttgcagcaagccgatatggaaagtaacggtaaattcatttgccgtgacggcagccgcgtcagctataaaactggaccagtcatctggggtgaagctggcaccaacggtcagcacgcattctatcagctgatacatcagggtacgcaaatggtgcccgctgatttcctgatgccagctaatagccattacaaagtaggtagcccggatgaccagcaccacaaaatcctgctggccaatttcctggctcaaacacaggcattgatgctgggtaagacccgtgaagaagcgcgtgcagaactcgaaaaacagggactgagtggcgaagcactggaagacttgttaccgcacaaagtattcgaaggtaaccgtcctacgacctccatcctgttcaaacagctgaccccttataccctgggcaagctgattgccctgtatgagcacaagatttttgtgcagggtattatctgggacatcaacagttacgaccaatggggtgtggaatacggcaagcaaatcgccagccaaatcttgccacaattgaatacaccagaagcggtgacagggtttgacagctcaaccaacggcttaatcaattacgcgaagaaaatatcgtaa

SEQ ID NO: 2 - protein, 550 aa, Methylophilus methylotrophus
MAKLTTHPSWHALNQHQIEMRNVQMRDLFKQNPKRFEEFSLTVEDVLLDYSKHRITKETLAHLFQLARDSNIEHWRSRMFSGEKINITENRAVLHTALRNRSNTPVYVDGKDVMPEVNAVLAQMRSFTEQVRSGQWTGYTGKRITDIVNIGIGGSDLGPVMACDALKPYASPDLKAHFVSNIDGAHLMRVLETCHPETTLFIVASKTFTTQETMTNAHSARRWFLNAAQQDQHVAKHFVALSTNAKAVQAFGIDTNNMFAFWDWVGGRYSLWSAIGLSIALYVGMDNFEQMLAGGHAMDQHFQTAPLEQNMPVIMALLGIWYNNFFHVDTHAILPYDQGMSRFAAYLQQADMESNGKFICRDGSRVSYKTGPVIWGEAGTNGQHAFYQLIHQGTQMVPADFLMPANSHYKVGSPDDQHHKILLANFLAQTQALMLGKTREEARAELEKQGLSGEALEDLLPHKVFEGNRPTTSILFKQLTPYTLGKLIALYEHKIFVQGIIWDINSYDQWGVEYGKQIASQILPQLNTPEAVTGFDSSTNGLINYAKKIS

SEQ ID NO: 3 - DNA, 1479 bp, Methylophilus methylotrophus
  CDS (1)..(1479)
atgacgaataaaattgatccatgcaccctggtattgtttggcgcgagtggcaacctggcccgcgtaaaactatatcctggtttgttcaggctagacatgctgggccgcctgccagatgacatgaaaattatcggcgttggccgccaagtggttgatctggacgcctggcgtgcagacatcaaatccatgctggataccaagtttaaaaaaggctatgaccagaaagtgttcgagcgttttatcgcccgtaacttctaccatgccaacccgccgaccgatccagatgctttcaaaaaactgaaagcgaccctgagcgacgaaaaagtattcccgcagaacctggcttacttcctgtctgtacgtcccgttgactttgccccagtggtggaatccctggcaaatgttggcctgactcaggaagacaaatactggcgtcgtgtagtgatcgagaaaccattcggtactgacctgccatctgcgaaagagctgcaggctgcgatcaccaagcacctcaaagaaagccaaatctaccgtatcgaccactacctgggtaaatctgccttgcagaacattctgctgcaacgttttaccaataccgtgttagagccgatctggaacaatcagtatatcgaccacgtacaaatcaccaacaccgaaatgctgggtgtaggcgaccgtacacagttctatgacagcactggcgcattgcgtgacatgctacaaagccatattttgcaaacattggctttaaccgccatggagcagccaaaagacctgtctcctgaaggcgtgcgtgatgcgaaaatcaaattgcttgaagaaattcgcccaattccagttaaagagctggaaaaacacgcattccgtgcacaatacgctgctggcgaaatcaacggcgaaaaagtccctggctacctggaagaattaggcaacaaggacagcgtcgttgaaacctatgcagcgttgaaactgtttatcgataatccgcgctggaaaggtgtaccgttctatatccgtacggcaaaacgcctgcacgaagccgacacacgcatttccatccgcttcaagaaggcacctttgcaaatcaatgaccaggaccagaactggctcattatcggcatccagcctcgcgagtgtatcaagctggaaatccagtccaagattcctggcctggacgtgcaaacacgtaccatccagctggatgcggctaaccgttgggatagcgacgacaccgtagatgcttacgaagccctgttgcttaacctgatgcaaggtgacaactccaactatctgcatatttctgaagctgaagcccaatggcgcctggtcgacccagtcgtgaaagcttgggcggaagacaagcgccctgtgcatcagtaccctgctggcagccgtgatccgattgaatccaaagtgatttttgaaggtgaagaccagttctggcgttacagcattgaactgggcggcgacaagttttag

SEQ ID NO: 4 - protein, 492 aa, Methylophilus methylotrophus
MTNKIDPCTLVLFGASGNLARVKLYPGLFRLDMLGRLPDDMKIIGVGRQVVDLDAWRADIKSMLDTKFKKGYDQKVFERFIARNFYHANPPTDPDAFKKLKATLSDEKVFPQNLAYFLSVRPVDFAPVVESLANVGLTQEDKYWRRVVIEKPFGTDLPSAKELQAAITKHLKESQIYRIDHYLGKSALQNILLQRFTNTVLEPIWNNQYIDHVQITNTEMLGVGDRTQFYDSTGALRDMLQSHILQTLALTAMEQPKDLSPEGVRDAKIKLLEEIRPIPVKELEKHAFRAQYAAGEINGEKVPGYLEELGNKDSVVETYAALKLFIDNPRWKGVPFYIRTAKRLHEADTRISIRFKKAPLQINDQDQNWLIIGIQPRECIKLEIQSKIPGLDVQTRTIQLDAANRWDSDDTVDAYEALLLNLMQGDNSNYLHISEAEAQWRLVDPVVKAWAEDKRPVHQYPAGSRDPIESKVIFEGEDQFWRYSIELGGDKF

SEQ ID NO: 5 - DNA, 687 bp, Methylophilus methylotrophus
  CDS (1)..(687)
atgtctttgaatgctcaaacccgttggctcacatttcaatcccagcaagaaattaatcaagctgccctgaacagaattttagctgctgctgatgcggcgattgccaaatatggcagctttctcattgtactagccggtggcagtacccccaaagccgtttatcagttgctggcaaaggctaatgccgactgggctaactggcatgtgtatcacaatgatgaccgctgcttgccggtcgatcatgccgaacgcaacagcaagatggcacgcgacgcctggttaagccatgtgcctatcccagaaaatcagattcacgatattcctgccgagctgggcaatgtggaaggcgccaaagcctatgctcaaactctcaaaggtgtgcgtacttttgacctggtgattcttggcctgggtgaagatggccataccgccagcctgttccctgggcaacttgtggacaactctgctgatgcggtccctgtctacaactcccccaaaccgcctgcagaccgtattaccatcagtcaacatcgcctcaacaatacacatgaagtcatgttcctggtgaccggggcaggtaaacaagaggcggtcgatcagtggcgtagcggggttgcaatcccggcaacattgattcaatcccccaatggtgtggatgtctattgttttggtgtcgagttgaaataa

SEQ ID NO: 6 - protein, 228 aa, Methylophilus methylotrophus
MSLNAQTRWLTFQSQQEINQAALNRILAAADAAIAKYGSFLIVLAGGSTPKAVYQLLAKANADWANWHVYHNDDRCLPVDHAERNSKMARDAWLSHVPIPENQIHDIPAELGNVEGAKAYAQTLKGVRTFDLVILGLGEDGHTASLFPGQLVDNSADAVPVYNSPKPPADRITISQHRLNNTHEVMFLVTGAGKQEAVDQWRSGVAIPATLIQSPNGVDVYCFGVELK

SEQ ID NO: 7 - DNA, 1815 bp, Methylophilus methylotrophus
  CDS (1)..(1815)
atgaaacaatctcacattgaagtaacagaacgcatcatcgagcgtagccgtccgacacgtaaggcgtatttgcagcgtattgatgattacctgaaccgcgaaaagggcccggatcgtctgggctgcgccaacgtggcgcacgcatttgcggctatgccacagaacgacaagttcaagatttacgccgaaaagcctacccatgtgggtatcgtcaccgcgtacaacgaaatgctgtctgcgcatcagccatatgtcaattatccagacattatccgtgatgaagcacgcaagcatggtgccactgtgcaggttgctggtggtgtgccagccatgtgcgacggcattacccagggtgagccaggcatggagttgagtctgttcagccgcgataccattgccatgagtactgctgtggctttatctcatgatgtatttgacgctgcattactgttgggcgtgtgtgacaagattgtgccggggctgctgattggtgccttgcactttggccatttgccttgtgtgtttgtccccgctgggccgatgagcaccggcattgataacacctccaaatccaaagtacgcgaaaaatacgcgcaaggtctgtgtgaccgtgatgaattgctagcaagtgagtcagcggcatatcacggcgcaggtacttgcaccttctacggaacagccaacagtaaccagatgctgctggaagccatgggtctgcatgtgccaggagcagcgtttattcacccgcatgatggcatgcgtgaggacttgacccgtgaggctgtgcgcttggtattgcaaaatgccaagaaagacaaatttgtgccgattggccgactggtagatgagcacgtgattgtgaatgccatggtggccttgctggctaccggtggttctaccaatcatttgatccactgggtagcagtcgcccgtgcagccggtatcatcattgactggactgatttccactacctggcaaaagccacaccgttactggcaagtgtgtatcctaacggtaaggctgacgtcaacgagttccagaccgctggaggtcctggttttgtgatccgtgaactgattgaggggggctacatgtaccctgatgtctacactgttgatggtggcgatttacgttcttatggcaaaaaacctgaaaaagaaggtgacaagctggtctggaaggactacccggcagaaagcggtgacgaaaccatcgtccgtaccgcagcacatccgttcaatgagtcaggtggcctgcgtttgcttaaaggtaatttaggccgtagcgtgatcaagatttctgcggtgccagaagaccgtcatattattgaagcgcctgcgattgtgtttgatgcacaggaagagctgctggcagccttcgaccgtggtgagctggaaaaagactttgttgccgtggtgcgtttccagggccctaaagccaatggtatgccagagctgcataaactgacaccaccgctggctgtattgcagaacaagggtttccgcgtagcgattgtgacagatggacgtatgagtggcgcttccggcaagatcccggcagcaattcacttgagtcctgaagcaagcgctggaggccccattgcgaaaatccgcaacggcgacattatccgcttgaatggtaccgtcggtcagttgaatgtgctggttgatgaagacacctgggctgagcgcgaggctgaagagttaagcgatgccaagcgtcattacaacgcacatggcttgggccgtgagttgtttggtggcatgcgcaggaatgtgttgtcagccgaagagggcgcagttacttggctgtaa

SEQ ID NO: 8 - protein, 604 aa, Methylophilus methylotrophus
MKQSHIEVTERIIERSRPTRKAYLQRIDDYLNREKGPDRLGCANVAHAFAAMPQNDKFKIYAEKPTHVGIVTAYNEMLSAHQPYVNYPDIIRDEARKHGATVQVAGGVPAMCDGITQGEPGMELSLFSRDTIAMSTAVALSHDVFDAALLLGVCDKIVPGLLIGALHFGHLPCVFVPAGPMSTGIDNTSKSKVREKYAQGLCDRDELLASESAAYHGAGTCTFYGTANSNQMLLEAMGLHVPGAAFIHPHDGMREDLTREAVRLVLQNAKKDKFVPIGRLVDEHVIVNAMVALLATGGSTNHLIHWVAVARAAGIIIDWTDFHYLAKATPLLASVYPNGKADVNEFQTAGGPGFVIRELIEGGYMYPDVYTVDGGDLRSYGKKPEKEGDKLVWKDYPAESGDETIVRTAAHPFNESGGLRLLKGNLGRSVIKISAVPEDRHIIEAPAIVFDAQEELLAAFDRGELEKDFVAVVRFQGPKANGMPELHKLTPPLAVLQNKGFRVAIVTDGRMSGASGKIPAAIHLSPEASAGGPIAKIRNGDIIRLNGTVGQLNVLVDEDTWAEREAEELSDAKRHYNAHGLGRELFGGMRRNVLSAEEGAVTWL

SEQ ID NO: 9 - DNA, 630 bp, Methylophilus methylotrophus
  CDS (1)..(630)
atgagcacattagagttagctaaccatggcccggtgattccggtcattgtcatcaacaaagtggaagatgctgtgcctatggcagaagccctgctggaaggcggtataaaggtgctggaagtgactttgcgttcaccagtggctttgcatgccatggaggaaattgccaaacatgtgccagatgcgattttaggttccggcaccgtgcgtaatctcaaggatgctaagaactcaaaagatgcaggttgccagtttgcagtgagtccaggctacaccagcgagttgggtaaatatgcccgtgaaattggtttgccattactgccaggcgtgtctacaggctcagagatcatgatggcgaatgcggatgattactacttcctgaaactgttcccagccgtagcggtgggtggcattaacctgcttaaaggctttgccgggccgtttgctgatgtgaaattctgcccaacaggtggcgtgagcgttgaaagtgcaccacagttcttggcactgccaaatgtggttgtgtgtggtggtacctggttgacgcctgcagatgccgtcgcaaacaaagattgggcgcatatcaccaagctggcgaaagaagcatcggcgattgtcgctgcgaaataa

SEQ ID NO: 10 - protein, 209 aa, Methylophilus methylotrophus
MSTLELANHGPVIPVIVINKVEDAVPMAEALLEGGIKVLEVTLRSPVALHAMEEIAKHVPDAILGSGTVRNLKDAKNSKDAGCQFAVSPGYTSELGKYAREIGLPLLPGVSTGSEIMMANADDYYFLKLFPAVAVGGINLLKGFAGPFADVKFCPTGGVSVESAPQFLALPNVVVCGGTWLTPADAVANKDWAHITKLAKEASAIVAAK

SEQ ID NO: 11 - DNA, 669 bp, Methylophilus methylotrophus
  CDS (1)..(669)
atggcgatggataaagcacaacaagacgcacttaaactggaagtggcgaaagcggctgtaacctatgtgaaagatggcattatcggcgttggcactggctcgactgccaacttttttattgatgaactggccaaggtcaaacacaaaattactggcgcagttgccagctccgaagccaccgcgcaacgcctgcgtaaccacggtatcgaagtattcgatttgaacagtgtagacagcctggatatttatgtagacggcgcggatgaaatcaccgaacacatgcatatgcttaaaggcggcggtggtgcgttgacccgagaaaagatcgtggcagctgtggccaaatctttcatctgtatctgtgacgaaagcaaattcgtacccgtcttgggcaagtttcccctgccagtagaagtattaccgatggcacgcagccatgtggcacgtgaactggtgaaactgggtggtcaaccacagttgcgtgacttcacgacggataacggcaacctgattctggacgtgcatggtttaaccatcagtgacccaatcgccatggaagccaaaatcaaccagatcgtcggcgtggtgaccaatggtttatttgctgcccgtccagccaatgtactcctgttggcgactccaaccggcgtcaaaacgctgacaaaataa

SEQ ID NO: 12 - protein, 222 aa, Methylophilus methylotrophus
MAMDKAQQDALKLEVAKAAVTYVKDGIIGVGTGSTANFFIDELAKVKHKITGAVASSEATAQRLRNHGIEVFDLNSVDSLDIYVDGADEITEHMHMLKGGGGALTREKIVAAVAKSFICICDESKFVPVLGKFPLPVEVLPMARSHVARELVKLGGQPQLRDFTTDNGNLILDVHGLTISDPIAMEAKINQIVGVVTNGLFAARPANVLLLATPTGVKTLTK

SEQ ID NO: 13 - DNA, 708 bp, Methylophilus methylotrophus
  CDS (1)..(708)
atggacaaaacttttcgcattgccccaagtattctttctgccaactttgccaaactaggccaggaaatcgaaaacgtcattaaatctggcacagacatcgttcactttgacgtcatggataatcactatgtgcctaacctgactatcggcccattggtctgtgatgcgattcgtgatctgtcacacaatgtaggcgccctgattgatgtgcacctgatggtcaaaccagtagaccgcatcattcctgactttgccaaagcgggtgctgacatcatcacattccacccggaagcctctgaccacattgaccgtagcctggccttgatccgcgatagcggttgcaagtctggcctggtatttaacccagccacaccgttgcattacctggattacgtaatggataaagtggacatgattttgctgatgtcggtgaacccggggtttggtggccagaaattcattccatccaccttggataaactgaaacaggcccgtgcccgcattgacgcttattacgaaaaaactggccgccagatctggctggaagtagatggtggcgtcaacgcaaacaacattgcagagatcgcaaaagccggggctgataccttcgtcgcaggtagtgcgattttcggttcacctaaagacaccgatcctaaccgttacgatacagttgtggcggcgatgcgtgcttccttagccactgtttaa

SEQ ID NO: 14 - protein, 235 aa, Methylophilus methylotrophus
MDKTFRIAPSILSANFAKLGQEIENVIKSGTDIVHFDVMDNHYVPNLTIGPLVCDAIRDLSHNVGALIDVHLMVKPVDRIIPDFAKAGADIITFHPEASDHIDRSLALIRDSGCKSGLVFNPATPLHYLDYVMDKVDMILLMSVNPGFGGQKFIPSTLDKLKQARARIDAYYEKTGRQIWLEVDGGVNANNIAEIAKAGADTFVAGSAIFGSPKDTDPNRYDTVVAAMRASLATV

SEQ ID NO: 15 - DNA, 1986 bp, Methylophilus methylotrophus
  CDS (1)..(1986)
atggcaactcgtgtcgacttgtgcaacgccatccgtgctctgagcatggacgctgtacaaaaagcaaactccggccacccaggcgcacctatgggcatggctgaaattgctgaagtattgtggaaccacaatctgagccacaacccaaacaacccacaatgggctaaccgtgatcgtttcgtattgtccaacggccatggctccatgctgatttactccttgctgcacctgactggttacgatgtgaccatggacgacatcaagtcattccgtcaactgcactcccgttgcgctggtcacccagagtacggctacgcacctggcgttgaaacaaccacgggtccgttgggccaaggtattgcgaacggcgttggttttgccatggcagaaaaactgctggctagccaattcaacaagccaggccacgacatcgttgaccactacacatacgtgttcctgggcgacggctgtatgatggaaggcgtttctcacgaagcttgtgcacttgctggtacatggggcctgggcaaactgatcgctttctgggatgacaacggtatttctatcgacggccacatcgaaggctggtacacagacgacaccgcaggccgcttcaaagcttacggctggcacgttgtttcagtagatggtcacgaccaggctgcgattcagaaagctatcgacgaagctaaatcagtgactgacaaaccatcactgatctgctgcaaaaccatcattggtaaaggttcaccaaacaagtgcggttcacacgactgccacggttccgcattaggcgaagctgaagttgctgcaacccgtgaagccatcggctggccacacgcaccttttgaaattcctgctgacgtatacgaaggctggaaccaaaaagacaaaggtgcaaaacgcgaagctgactggaacgcgaagtttgacgcttacgctaaagcctacccagcagaagctgcagaattcaaacgccgtatggccggtgaattgccagctaactggaagtcactgactgacgcaatcatcgctgaaaccaacgaaaaagctgagaaattggctactcgtcaagcttcacaaaaagcaattacagcgttggcgccaatcttgccagaattcctgggcggttcagctgacttgacaggttctaacctgacatcctctaaagacttcaaacacgtgagtggtaaagagccaggcaactacatctcttacggtgtacgtgagttcggtatggctgcaatcatgaacggtatggcattgcacggtggcttgttgccttacggcggtaccttccacatgttctctgactacatgaaaaacggcatgcgtatgtctgcgttgatgcatcaacgtgtgatctacgtgctgacccatgactctatcggtcaaggtgaagatggtcctacacaccaaccagttgaaaacactgctggtctgcgtatgattcctcgtatggatgtatggcgtccagctgactctacagaaacaaccgttgcatgggttgctgcagtagagcgcactgaaggcccaaccagcttggtattgagccgtcaagctgtgccaggcatcaagcacgatgctaaagactttgaactgatccgcaagggtggttatgtattctcagacgctgcaggtaaagcggatgtgatcatcattgctaacggttccgagttggatctggcgattcaagcggctgctgaattgaacgctgcgggtactaaagtgcgtgtggtttccatgccatccaccaacgtattcgaccgtcaagaccaggcttacaaagacagcgtattgactccaggcgttaaacgcgtggctgttgaagctgctcacccagatttctggcgtaagtatgttggtctggaaggtgcagttgttggtatcgataccttcggcgaatccgcaccaggcggcgcactgttcaaacacttcggcttcacagttgaaaacgtagtcaacacagttaaatccgtactgtaa

SEQ ID NO: 16 - protein, 661 aa, Methylophilus methylotrophus
MATRVDLCNAIRALSMDAVQKANSGHPGAPMGMAEIAEVLWNHNLSHNPNNPQWANRDRFVLSNGHGSMLIYSLLHLTGYDVTMDDIKSFRQLHSRCAGHPEYGYAPGVETTTGPLGQGIANGVGFAMAEKLLASQFNKPGHDIVDHYTYVFLGDGCMMEGVSHEACALAGTWGLGKLIAFWDDNGISIDGHIEGWYTDDTAGRFKAYGWHVVSVDGHDQAAIQKAIDEAKSVTDKPSLICCKTIIGKGSPNKCGSHDCHGSALGEAEVAATREAIGWPHAPFEIPADVYEGWNQKDKGAKREADWNAKFDAYAKAYPAEAAEFKRRMAGELPANWKSLTDAIIAETNEKAEKLATRQASQKAITALAPILPEFLGGSADLTGSNLTSSKDFKHVSGKEPGNYISYGVREFGMAAIMNGMALHGGLLPYGGTFHMFSDYMKNGMRMSALMHQRVIYVLTHDSIGQGEDGPTHQPVENTAGLRMIPRMDVWRPADSTETTVAWVAAVERTEGPTSLVLSRQAVPGIKHDAKDFELIRKGGYVFSDAAGKADVIIIANGSELDLAIQAAAELNAAGTKVRVVSMPSTNVFDRQDQAYKDSVLTPGVKRVAVEAAHPDFWRKYVGLEGAVVGIDTFGESAPGGALFKHFGFTVENVVNTVKSVL

SEQ ID NO: 17 - DNA, 954 bp, Methylophilus methylotrophus
  CDS (1)..(954)
atggcaaacttattagatcagttgaaagcaatgacaaccatcgtggctgacactggtgatgttgaagcaatcaaaagtgttaaacctgtggatgcaaccacaaacccatctctggtgttgaaagccagccaaatcccagaatacgcaccgttgattgacactgccattgcttatgcaaaagcacaaggtggttccaaagagcaacaaattgaaaatgctgctgacaaactggcagtattgattggcgctgaaatcactaaagtggttccaggccgtatttctacagaagtagatgcacgtttgtctttcaacatcgatgcgatgattgctaaaggccgtaaattgatcaagttgtacgaagagtctggcatcagcaaagaccgcgtactgatcaaacttgcttctacctgggaaggtatcaaggctggtgagcagctggaaaaagaaggtatccagtgtaacctgacattgttattcggtttcggccaggcgcgcgcatgtgctgaagctggcgtgttcctgatctccccattcgttggccgtatcctggactggtacaaagcgaaaaatccagggactgagtacactcaagaaactgacccaggtgtagtttctgttcgtgcaatctaccagtactacaaagagcacggctacaaaaccgttgtgatgggtgcttctttccgtaacactggtgaactgattgcgctggctggttgtgaccgtttgaccgtgtccccaaacctgctgcaagaattggcagcgacagaaggtacattggctcaagtgttgaaagacggtggcaagactaaagacgtgccagctaaaatgactgaagaagaattccgcttccagttgaatcaggacgccatggcgactgaaaaactggcagaaggtattcgcggtttcgtggctgaccagaaaaaactggaagacgctttagccgcaaaactgtaa

SEQ ID NO: 18 - protein, 317 aa, Methylophilus methylotrophus
MANLLDQLKAMTTIVADTGDVEAIKSVKPVDATTNPSLVLKASQIPEYAPLIDTAIAYAKAQGGSKEQQIENAADKLAVLIGAEITKVVPGRISTEVDARLSFNIDAMIAKGRKLIKLYEESGISKDRVLIKLASTWEGIKAGEQLEKEGIQCNLTLLFGFGQARACAEAGVFLISPFVGRILDWYKAKNPGTEYTQETDPGVVSVRAIYQYYKEHGYKTVVMGASFRNTGELIALAGCDRLTVSPNLLQELAATEGTLAQVLKDGGKTKDVPAKMTEEEFRFQLNQDAMATEKLAEGIRGFVADQKKLEDALAAKL

SEQ ID NO: 19 - DNA, 1017 bp, Methylophilus methylotrophus
  CDS (1)..(1017)
gtgacatgtggccacatttgcttaatccataaaggtattgccatgcaattggacgtgtttttaaaaaaggtttctatcaatcatgcccttaatacgctgatctggcatattgccgaggcgggcattgagattgctgcgctggtcaggcagggcgcattggcgggggtcaccgaaaaaatgaccagtaccaatgtgcagggtgaaacacagatgcatctggatatccgtagtcacgaagtggcgctggcccagttacggacctctgcagtggtagctgctgtgttatctgaagaagaggatcagcctgtgttgtttgaaacattggaggcgcctttcctggtcagcatggatcctttggacggttcttccaacctggcgattaacggtgtggtgggtagtattttctcggtattgcccaatactggcctggcaacccttggcgagcaggcttttttacagccgggcaaggatcagcgtgcggcggcttatatcatgtatgggccagcgactttgctggtgctgacgattggtcagggcacgcatgtgtttacattggaccatagcagccaggcgtttgtattgacacaacaaacagtcaaagtccccgaagaaaccacagagtttgccatcaatgccagtaatcagcgttattggcaacccgccatgcagcgctatattatggaatgtctgcaaggtttagaagggccgcgtggacgcgactttaatatgcgctggtgtgcaagtatggtgatggatgtacatcggatcctgacgcgtggaggggtgttcctttatccgcgtgatagcaagcaaccggtgaaagccgggaggttaagattgttgtatgaagccaaccccatgagcttgctggtgacgcaggcgggcggcgaaagtatagacgggctgcaagaggttctgaccatcgcaccggatagctgccatcagcgcgtgccagtgttgctaggctcaaaacaggaaattcagcgcttgcgcgactatcatcagcaagccgattag

SEQ ID NO: 20 - protein, 338 aa, Methylophilus methylotrophus
VTCGHICLIHKGIAMQLDVFLKKVSINHALNTLIWHIAEAGIEIAALVRQGALAGVTEKMTSTNVQGETQMHLDIRSHEVALAQLRTSAVVAAVLSEEEDQPVLFETLEAPFLVSMDPLDGSSNLAINGVVGSIFSVLPNTGLATLGEQAFLQPGKDQRAAAYIMYGPATLLVLTIGQGTHVFTLDHSSQAFVLTQQTVKVPEETTEFAINASNQRYWQPAMQRYIMECLQGLEGPRGRDFNMRWCASMVMDVHRILTRGGVFLYPRDSKQPVKAGRLRLLYEANPMSLLVTQAGGESIDGLQEVLTIAPDSCHQRVPVLLGSKQEIQRLRDYHQQAD

SEQ ID NO: 21 - DNA, 1035 bp, Methylophilus methylotrophus
  CDS (1)..(1035)
atggcattgatctcactcaggcagttgctggaccacgctgcggaacatagttatggttacccggcattcaatatcaataatatggagcagatactctccatcatgaaagccgctgatgaagtagatagtgcggtgattttgcaggcgtcggctggtgcaagaggctatgctggtgagtcattcctgcgcaagatggtggaagctgccattgagcaatacccgcatattccagtctgtatgcaccaggatcatggaacttcgcccaagatttgccagatggcgatacgcagcgggttttccagcgtgatgatggatggctctttgaaagaagatcataaaacgccagccagttatgattacaatgtggatgtcacgcgtcgtgttgtcgagtttgcccatgcggtgggggtttcggtagaaggcgagcttggtgtactcggctcgcttgaaactggcatggcaggcgaggaggatggggtaggggcagaaggtaagctggatgaatcccaattgctgaccgaccccgacgaagccgctgcttttgttgaagcgaccaaggtcgatgcactggccattgccattggcaccagtcatggcgcctataaattcacgcgcccaccttcagctgatacgctgtcgattgaacgtatccgggaaattcatgccaaaatccctaacacgcacttggtgatgcatggttcttcgagtgtgccgcaatccttgctggaacaaatcaggcactatggcggcaatatcaaggaaacttacggtgttccggtttcgcaaattgtcgaagggattaaaaacggcgtgcgtaaggtcaatattgataccgatatccgcctggccatgaccgctgctatccgtgcgcatttggcggagtatccagagcaatttgatccgcgacaatattttaaagaagctactattgcagcccagcatttgtgtaaagaacgcttcgaggcctttggtagtgccggacaagcgagtaagataaaggttgttccacttgaaaaaatggcagccatctactaa

SEQ ID NO: 22 - protein, 344 aa, Methylophilus methylotrophus
MALISLRQLLDHAAEHSYGYPAFNINNMEQILSIMKAADEVDSAVILQASAGARGYAGESFLRKMVEAAIEQYPHIPVCMHQDHGTSPKICQMAIRSGFSSVMMDGSLKEDHKTPASYDYNVDVTRRVVEFAHAVGVSVEGELGVLGSLETGMAGEEDGVGAEGKLDESQLLTDPDEAAAFVEATKVDALAIAIGTSHGAYKFTRPPSADTLSIERIREIHAKIPNTHLVMHGSSSVPQSLLEQIRHYGGNIKETYGVPVSQIVEGIKNGVRKVNIDTDIRLAMTAAIRAHLAEYPEQFDPRQYFKEATIAAQHLCKERFEAFGSAGQASKIKVVPLEKMAAIY

SEQ ID NO: 23 - DNA, 795 bp, Methylophilus methylotrophus
  CDS (1)..(795)
atgtcccgttctgatacaacccttccttccccacacaaaattgtggttgccaactggaaaatgcatggcaatcttgcccgtaaccacacgcttgttgatggctatctgcaaggcttgaaatcattgtcccaagccgatgtcgttatctgtgtgccttatccttatctggcgcaaatacagtctttgttgtctgagactcacgtcgcctggggtgcgcagaatcttgccaagtacgaggaaggtgcttataccggtgaggtgagcgcggggatgttgtgcgattttggggcgcaatatgtgattatcggccactccgagcgaagtactgcttattgcgagtcggatgaaaatattgccgaaaaattcatgatggccaagcggcatgggttaacgccaattctgtgtgttggagagaccttgcttgaacgcgaggctggtgtcatggagcgtgttgtcggtaaacaactggaaaccattattcgtctgtttggcggagaggccttcgcaaatagcatcgtttcatatgagccgatttgggcaattggaaccggccttgcagcttcagcagagcaggcggtggctatgcaccagtttatccgtgacaccgtatcagccgcagataagagcgccgcggacacgctaaaaatcctctacgggggcagcgtaaacccgcaaaatgcggtacaattattaaatcagcaggaaattgatggcgcgctggtcggccgttgctccctcaatgcggagcaatttataaaaatctgtcaggccgtccctgaaaatagcgtcgtctag

SEQ ID NO: 24 - protein, 264 aa, Methylophilus methylotrophus
MSRSDTTLPSPHKIVVANWKMHGNLARNHTLVDGYLQGLKSLSQADVVICVPYPYLAQIQSLLSETHVAWGAQNLAKYEEGAYTGEVSAGMLCDFGAQYVIIGHSERSTAYCESDENIAEKFMMAKRHGLTPILCVGETLLEREAGVMERVVGKQLETIIRLFGGEAFANSIVSYEPIWAIGTGLAASAEQAVAMHQFIRDTVSAADKSAADTLKILYGGSVNPQNAVQLLNQQEIDGALVGRCSLNAEQFIKICQAVPENSVV

SEQ ID NO: 25 - DNA, 783 bp, Methylophilus methylotrophus
  CDS (1)..(783)
atgacaaatcgctccaagctggtggttgccaaccgcaaaatgcacggcaacttgccagacaatcagcagtttatgcaaacgctattgcaacaaacccgccatcataaggcacgttatgcggtatgcccgccgcatccgtacctgttccaggcacagcaagtgctgcaagacagccatattgcctggggtgggcaaaacatgagccgctatgaaaaaggcgcctatacgggctcggtctcaccgctcatgctcaaggagtttggctgcagctatgtgattatcggtcactcagaacggcgtcaacgcggtcacgatagtgatgaaacctgcggagaacgttttgaagccgcgctcaaggccggcctcacgcccattttgtgcatcggtgaaaccttggaagaatacgagcaaggcgaaactgaccttgtcgtggtacggcaactcaacccggtgattgcgcatgtcggcatcgaggcgctatcaaaaggcgtgattgcctatgagccagtgtgggccataggtacaggcagagcagccactccacagcatgcacaagccattttgtcttttatccgtggccatattgaattactggacacaaaagccgccgaggacattaccttgctgtatggtggcagtatgaatcctggcaatgccgcacaactattgagcatgcccgatgtagatggcggcctgatcggcggcgcctccctggtagcagacgactttatccgcatctgccagattgccaacgaattgaccccgctcaaaaccgcagcctga

SEQ ID NO: 26 - protein, 260 aa, Methylophilus methylotrophus
MTNRSKLVVANRKMHGNLPDNQQFMQTLLQQTRHHKARYAVCPPHPYLFQAQQVLQDSHIAWGGQNMSRYEKGAYTGSVSPLMLKEFGCSYVIIGHSERRQRGHDSDETCGERFEAALKAGLTPILCIGETLEEYEQGETDLVVVRQLNPVIAHVGIEALSKGVIAYEPVWAIGTGRAATPQHAQAILSFIRGHIELLDTKAAEDITLLYGGSMNPGNAAQLLSMPDVDGGLIGGASLVADDFIRICQIANELTPLKTAA

SEQ ID NO: 27 - DNA, 825 bp, Methylophilus methylotrophus
  CDS (1)..(825)
atgcgacaaaaattagtcattggtaactggaagctgcatggcggcctgcttgaaaatcagggcctgttgaatcgtctcaagcaagagttgcacgatctacctggcgtggatgccgcggtgtgtttaccctatgtatacctgttccaggcacaaaccctgctacaagattcccatattgcctggggcgcccaaaacgtcagtcaattcaccgaaggtgcgtttacctcctgcatttcagccaaaatggtagccgaatttggctgcacctataccatcattggccattctgagcgtcgcgccctcaagctcgaaagtaaccaggtcgccaccaaacgcctgcttaatgccctgcatgccgggctcacgcctattttctgtgtcggtgaaacgcaggatgaacgcgatggcaatatggctgagctgattgtgcgcaaccagatgctgaatgtcgtctacggtctggatgatgaagcgtttgcccttgccaaaaaatttaatatggtgattgcctatgaaccggtatgggccattggtacgggcgagcatgccagcccggaacaggcgcaacgcatgcatgcctttatccgcatgatgattgccgagcgcgaccgcgagtttgcagatcgcatccgtattgtctatggtggcagtatgaccccaaaaaacgcgcacagcctgctcagcatgccggatatcgatggtggtttgctgggccgcgcagccttggtggcagaagattttgttgaaatctgcagaatttccagccgttgctacatgcaaaaaacagcgttcagggaaccctcgcccgtcatgccagtcatttga

SEQ ID NO: 28 - protein, 274 aa, Methylophilus methylotrophus
MRQKLVIGNWKLHGGLLENQGLLNRLKQELHDLPGVDAAVCLPYVYLFQAQTLLQDSHIAWGAQNVSQFTEGAFTSCISAKMVAEFGCTYTIIGHSERRALKLESNQVATKRLLNALHAGLTPIFCVGETQDERDGNMAELIVRNQMLNVVYGLDDEAFALAKKFNMVIAYEPVWAIGTGEHASPEQAQRMHAFIRMMIAERDREFADRIRIVYGGSMTPKNAHSLLSMPDIDGGLLGRAALVAEDFVEICRISSRCYMQKTAFREPSPVMPVI

SEQ ID NO: 29 - DNA, 1002 bp, Methylophilus methylotrophus
  CDS (1)..(1002)
atgtcagtcaaggtaggcattaacgggtttggacgaattggccgtatggcgttacgtgcggcgattgagcaagcagagttcagcaatatcgaagtggtcgctattaatagctcatatgatgttgaatacatgatgtatttgcttaaatatgactctgtgcatggccgttttaatgctaaggttgaagctgacaatggtgcgctggtggtgaatggcaagcgtatccatttaacggcagagcgagatccaaacaatattgactggcgtaaaggaggggctgaggtggtgattgaatctaccggtgcctttttaacgcaggacaactgccagccgcacctcaacggtggtgccatcaaagtggtgcagtctgcacccggcaaagacgatacgccgatgtttgtctatggcgtgaaccacaccgaatatgctggccaggcaattatttcagcagcatcttgcactactaacgggttggcacccctggcaaaagtgttgcatgatacctttggtgtcaagcgcggcctgatgaccacgattcatgctgccacggcatcccaactgaccgtagatggcacctccaaaaaagactggcgcggcggacgcagtgtctttgaaaacattattccttcgagtacgggtgcagccaaggcagtgggtaaggtaatcccggccttgaacaagaagttaacagggatgtccatgcgcgtgccttctgcggatgtgtcggtggttgacctgaccgtggaacttaatagcgaggctacttatgaggcgatttgcacagccatgcaacaagcggccgatggcccgctaaaaggcgtgctggagtataccaatgacaaagtcgtttccagtgacttccgcagcagccctgctgccagcgtgtttgatgcagatgccggtatcatgctcgacccgacttttgttaaagtggttggctggtacgacaacgagtacggttatacctgtaacctgctgcgcctggttcaacacattgcttaa

SEQ ID NO: 30 - protein, 333 aa, Methylophilus methylotrophus
MSVKVGINGFGRIGRMALRAAIEQAEFSNIEVVAINSSYDVEYMMYLLKYDSVHGRFNAKVEADNGALVVNGKRIHLTAERDPNNIDWRKGGAEVVIESTGAFLTQDNCQPHLNGGAIKVVQSAPGKDDTPMFVYGVNHTEYAGQAIISAASCTTNGLAPLAKVLHDTFGVKRGLMTTIHAATASQLTVDGTSKKDWRGGRSVFENIIPSSTGAAKAVGKVIPALNKKLTGMSMRVPSADVSVVDLTVELNSEATYEAICTAMQQAADGPLKGVLEYTNDKVVSSDFRSSPAASVFDADAGIMLDPTFVKVVGWYDNEYGYTCNLLRLVQHIA

SEQ ID NO: 31 - DNA, 1062 bp, Methylophilus methylotrophus
  CDS (1)..(1062)
atgggcattagagtgggcattaacgggtttggccgtattggccggatggtgctgcgcgccgcattaaatcaggcagagttcaaggatatcgaggttgttgccattaacggcatcgaagaacctgaacatatgctgtatatgctcaaatatgactctgtgcacggaagactggcacaggaagctcatttagagggcggtttcctggtgattgatggtaaaaaaatccgtctcactgcacatgccaaaccggcagacattgactggcggtctgagcatgtcgatgtcgtggtggaatgtacgggtgttttcctgacccaggaaagctgtgctgcccatattgcaggtggtgcacgtatcgtagtccagtccgcgcctggcaaagacgatacgccaatgtttgtctatggtgtcaatcattaccactaccgcggagaagatattgtctcggcagcgtcttgtacgactaatggcttggcgcccattgttaaggtgttacatgacaattttggggtcaagcgtggcttgatgactaccatacacgcagcgactgccacccagaaaacggttgatggtacttctaacaaagactggcgtggcggtcgaggtgttttcgataacattattccttccagcactggtgcggccaaggcggttggtaaagtaatcccggcacttaacaaaaaactcaccgggatgtccatgcgtattccttccgcggatgtgtctgtggttgatttaacggtagagttgcaacagagcaccacttatgaagaaatttgcgccgtcatgcaatatgcagccgagcgcgacctgaaaggcgtgctaggctataccgatgaagccgtcgtttccagtgattttaggggttatccggccgccagtgtgtttgatgccagtgcaggcatcatgctggaccccacctttgtcaagctgataggctggtatgacaacgagtatggctatacctgcaatctgttgcgcttaacgcgttatgtcggttatgccaagctgtcgatgacggatttgccgcaaactgagcagcaagaggccgttgccgcttga

SEQ ID NO: 32 - protein, 353 aa, Methylophilus methylotrophus
MGIRVGINGFGRIGRMVLRAALNQAEFKDIEVVAINGIEEPEHMLYMLKYDSVHGRLAQEAHLEGGFLVIDGKKIRLTAHAKPADIDWRSEHVDVVVECTGVFLTQESCAAHIAGGARIVVQSAPGKDDTPMFVYGVNHYHYRGEDIVSAASCTTNGLAPIVKVLHDNFGVKRGLMTTIHAATATQKTVDGTSNKDWRGGRGVFDNIIPSSTGAAKAVGKVIPALNKKLTGMSMRIPSADVSVVDLTVELQQSTTYEEICAVMQYAAERDLKGVLGYTDEAVVSSDFRGYPAASVFDASAGIMLDPTFVKLIGWYDNEYGYTCNLLRLTRYVGYAKLSMTDLPQTEQQEAVAA

SEQ ID NO: 33 - DNA, 1194 bp, Methylophilus methylotrophus
  CDS (1)..(1194)
atgaaatttatcaaaatgactgacctcgacttgcgtggtaaacgtgtgttcatccgcgccgacctcaatgtgccggtaaaagatggcgtggtgaccagtgatgccagaattgtagccagcatgcccaccattgaatacgccttgaaagcgggcgctaaagtcatggtgacttcgcatcttggccgcccagaagaaggcgtttacagtgaagaaaactcattgaggccagtggcggatgtaatgagcaagctgctggggcagccagtacgcctggtgaaaaactggctggcccccaatgcgcctgaagacagcctgaccacagcagatggtcagctcattttgctggaaaactgccggtttaatgtaggcgaaaagaaaaataatgatgatctggccaaaaaatacgccgccttgtgcgatgtatttgtcatggatgcttttggcactgcacaccgcgcagaagcctctacacatggcattgccaaatttgcgcctgtggcctgtgccggaattctgctcactgaagagctggatgcattgagcaaagccttgctggcgcccgcccgcccgctggtagcgattgtggggggctcaaaagtctcgaccaaactgaccgtgcttgaaagtttgtctgacaaagtggatcagttggtggtaggcggcggtattgctaacaccttcctcaaggcttctggtcaggaagtcggtaagtctttgtgtgaggatgacctggtggataccgcgaagtgcctgatggataaaatggcggcgcgtggtgcctgtgtgcctattgctaaagatgtggtggttggcaagcagtttgatgccaatgaacctgctgtcaaaaaatcagcaacagaagttgccgtagacgaaatgatttttgatattgggtcacaatcagcagctgagctggtggacatcattatgaaagctggcactgtcgtatggaatggtccggtgggtgtgtttgagtttgaccagtttggtgaaggtactaaaaccattgccaaggccatcgctgaaaccaaggcctttacgctggcaggtgggggtgacaccattgcggccatccagaagtacgacatttatgacaaggtatcttacatctcgacggctggcggggctttcctggagtttctcgaaggcaagactctgccggctgtggctattctggaagagcgcgcccgttaa

SEQ ID NO: 34 - protein, 397 aa, Methylophilus methylotrophus
MKFIKMTDLDLRGKRVFIRADLNVPVKDGVVTSDARIVASMPTIEYALKAGAKVMVTSHLGRPEEGVYSEENSLRPVADVMSKLLGQPVRLVKNWLAPNAPEDSLTTADGQLILLENCRFNVGEKKNNDDLAKKYAALCDVFVMDAFGTAHRAEASTHGIAKFAPVACAGILLTEELDALSKALLAPARPLVAIVGGSKVSTKLTVLESLSDKVDQLVVGGGIANTFLKASGQEVGKSLCEDDLVDTAKCLMDKMAARGACVPIAKDVVVGKQFDANEPAVKKSATEVAVDEMIFDIGSQSAAELVDIIMKAGTVVWNGPVGVFEFDQFGEGTKTIAKAIAETKAFTLAGGGDTIAAIQKYDIYDKVSYISTAGGAFLEFLEGKTLPAVAILEERAR

SEQ ID NO: 35 - DNA, 1551 bp, Methylophilus methylotrophus
  CDS (1)..(1551)
atgcctatcaaacctgttattttgctgattctcgatggttttggtcacagcgagaccgttgaattcaatgccgtcctgcaagcgaacacccccaacctggacagacttagagcgacgtacccgcatagcttgatcaatgcctctgaacactacgttggcctgccggacggacagatgggcaactcagaagtgggccacttgaatattggggcgggacgtatcgtctttcaagacttcgagcgcattaataacagcatacacaccggtgagtttttccagttacccgcactggtgaatgccatgcaagacctcaaagccaacgacaaagccctgcacatcctgggcctgctgtctgacggtggcgtacacagctaccagccacacattcacgccatgctcgatatggccaaacagcaaggcctgaacaaagtgtatgtgcatgccttcctagatggccgtgacacaccgccaaagagcgcgcaaccttacctgcaagcactcgaagatcacctgaaaacccttggcgtaggcaaagtggcctctgtgggtggccgtttctatggcatggacagggacaaacgctgggagcgcgtttcggtcgcgtatgagttactggtcaatggcattgcagaccatgttgcacccgatagcctcaccgcattactacaggcttatgcgcgtgatgagagtgacgagtttgtcaaatgcaccgccatccgcgccgccaatgacgccccgatccggatggaggacggtgactgcctggtgtttatgaacttcaggagtgatcgcgcccgccaattgactgacgccttgctcaaccagcaatttaccggtttttcccgtagccgcatccctcacttcagtcactactttacgctgacccagtatgacaaaaaccagacgctggctgaaccgatttttgccccctacacggttcctaacacgtttggtgagtacgtctccaagcttggcctcaaacaactacgtatcgccgagaccgagaaatatccgcacgtgacatttttcttcaatggcggtgaagaaacggtatttgatggtgaagaccgtatcctggtcccttcgcctaaagtcgccacttacgacttgcaacctgaaatgagcgcacctgaagtcaccgacaaactggtggccgcaattgagtctgggcaataccaggcggtgatatgcaactatgccaatggcgacatggtcggccataccggcaacctgcaggcggcgattaaagctgtggaaacactggatacctgtattggccgtgtggtagccgctgcgcagaaaatgggtgcagaagtgattattaccgcagaccacggcaatgcagaaagcatgtttgaccatgccagcgatcaggcacacacgcagcacaccaccaacctggtgccgtttatttatattggccgcccagggagcattgctgcaggtggtgcgctttctgatattgcgccgacgttgctgtcactgatgggcattccccaaccttcagaaatgacaggcaagaacttgatcaccctcaatgcaatggcttaa

SEQ ID NO: 36 - protein, 516 aa, Methylophilus methylotrophus
MPIKPVILLILDGFGHSETVEFNAVLQANTPNLDRLRATYPHSLINASEHYVGLPDGQMGNSEVGHLNIGAGRIVFQDFERINNSIHTGEFFQLPALVNAMQDLKANDKALHILGLLSDGGVHSYQPHIHAMLDMAKQQGLNKVYVHAFLDGRDTPPKSAQPYLQALEDHLKTLGVGKVASVGGRFYGMDRDKRWERVSVAYELLVNGIADHVAPDSLTALLQAYARDESDEFVKCTAIRAANDAPIRMEDGDCLVFMNFRSDRARQLTDALLNQQFTGFSRSRIPHFSHYFTLTQYDKNQTLAEPIFAPYTVPNTFGEYVSKLGLKQLRIAETEKYPHVTFFFNGGEETVFDGEDRILVPSPKVATYDLQPEMSAPEVTDKLVAAIESGQYQAVICNYANGDMVGHTGNLQAAIKAVETLDTCIGRVVAAAQKMGAEVIITADHGNAESMFDHASDQAHTQHTTNLVPFIYIGRPGSIAAGGALSDIAPTLLSLMGIPQPSEMTGKNLITLNAMA

SEQ ID NO: 37 - DNA, 1287 bp, Methylophilus methylotrophus
  CDS (1)..(1287)
atgagcgctattgtagatattatcgcccgcgaaattatggattcacgcggcaacccgacggttgaagtggacgtattgctggaaagcggtgtgattggtcgtgcggctgtgccttctggcgcttccacaggcactaaagaagcggttgaactgcgtgacggcgataaaagccgttatttaggtaagggtgtattgaatgcggttgaaaacgtcaatactgaaatcactgaagcgatcattggcctagatgctgaagagcaaagttttattgataaaacgctgattgagctggacggcacagaaaacaaggaccgcctgggcgcgaatgcaatacttggcgtgtccatggcttgtgcccgtgccgcagctgaagagagtggcctgccgttgtatcgctatctgggcggttccgcgttcatgcagttgcctacgccgatgatgaacatcatcaacggtggtgcacatgcggacaactccgtagatattcaagagttcatgattgtgccagctggtttgccaacttttcgtgaggccctgcgcgcaggtgcagaagtgttccatgccttgaagaaaaccctgcatgccaagggcttggctactaccgtaggtgatgaaggcggttttgcgcctaacctgccatccaatgaatctgccctgcaattgatcatggaatcgattgaagctgcgggttatgagccaggcaaagacatctacctgggcctggattgcgccagtaccgagttttacaaggatggtaaataccatctggagtcagagggtgcatcgctgacttcacaacagttttcagattacctggcgacattggctgacaaataccccatcattactattgaagacgggatgagtgagtttgactgggagggctgggatatcctgaccaaacgcctgggtaaaaccacgcagctggtgggtgacgacctgtttgtgactaaccctaaaatcctgcgtgaaggcatccagaagggcgctgccaactcagtgctgatcaaggtgaaccagattggtaccttgaccgaaaccttccagactatcgaaatggctaaacgtgcaaactacaccgctgtcgtttcacatcgttctggtgagactgaagatacaacgattgctgatatctctgtcgccaccaacgcgttgcagatcaagaccggttcattgtgccgttccgagcgcgtagccaagtacaaccagttgctgcgtattgaagaagaactgggcgatgcaaccagctatgctggcttgtctgctttctaccagttattcaagtaa

SEQ ID NO: 38 - protein, 428 aa, Methylophilus methylotrophus
MSAIVDIIAREIMDSRGNPTVEVDVLLESGVIGRAAVPSGASTGTKEAVELRDGDKSRYLGKGVLNAVENVNTEITEAIIGLDAEEQSFIDKTLIELDGTENKDRLGANAILGVSMACARAAAEESGLPLYRYLGGSAFMQLPTPMMNIINGGAHADNSVDIQEFMIVPAGLPTFREALRAGAEVFHALKKTLHAKGLATTVGDEGGFAPNLPSNESALQLIMESIEAAGYEPGKDIYLGLDCASTEFYKDGKYHLESEGASLTSQQFSDYLATLADKYPIITIEDGMSEFDWEGWDILTKRLGKTTQLVGDDLFVTNPKILREGIQKGAANSVLIKVNQIGTLTETFQTIEMAKRANYTAVVSHRSGETEDTTIADISVATNALQIKTGSLCRSERVAKYNQLLRIEEELGDATSYAGLSAFYQLFK

SEQ ID NO: 39 - DNA, 906 bp, Methylophilus methylotrophus
  CDS (1)..(906)
atgaaattagcaatgattggcttgggcaaaatgggcggcaacatggctgcacgcttgctgcgtcacaaaatcaacgtagttggctttgacttcaacaccgaattcgttaacagactggttaaagaagaaggcatggaagcggcaacctctgtggcggatgctgtaagcaaactcgaaggccaaaaaattgtctggctgatgttgcctgctggcgaaatcaccgaaaatcaaatcaaagatttgattccaatgctgaacaaaggcgacatcatcgtcgatggcggcaactctaactacaaacacagtcagcgtcgcggtgcaatgttggctgagcacggtatcggctttatcgattgcggtacttctggcggtgtttggggccttgaaaacggctactgcctgatgtacggcggtgaaaaacaatatgcagatgtattggcaccttacgcacaggcactgactcacgcagaccgtggttgggcacacgtgggtccagtcggctccggccacttcacaaaaatgatccataacggtatcgaatacggcatgatgcaagcgtttgctgaaggtctggacctgatcaaaggtaaagaagacttcaacctggatctggcacaaatcactgaattgtggcgtcatggttccgtggttcgcagctggttgctggatctgactgctgaagcactgaaaggtgaccagaagctggaagccatcgctccttatgttgctgactccggcgaaggccgctggacagtggttgaagcggttgaacaaggtgttgctgcgccagtactgactgttgcattgcaagcccgtttccgcagccaggactccaaaggttacagctacaaactgttgtcactgatgcgtaatgcatttggtggccacgcagttaaaaccaagtaa

SEQ ID NO: 40 - protein, 301 aa, Methylophilus methylotrophus
MKLAMIGLGKMGGNMAARLLRHKINVVGFDFNTEFVNRLVKEEGMEAATSVADAVSKLEGQKIVWLMLPAGEITENQIKDLIPMLNKGDIIVDGGNSNYKHSQRRGAMLAEHGIGFIDCGTSGGVWGLENGYCLMYGGEKQYADVLAPYAQALTHADRGWAHVGPVGSGHFTKMIHNGIEYGMMQAFAEGLDLIKGKEDFNLDLAQITELWRHGSVVRSWLLDLTAEALKGDQKLEAIAPYVADSGEGRWTVVEAVEQGVAAPVLTVALQARFRSQDSKGYSYKLLSLMRNAFGGHAVKTK

SEQ ID NO: 41 - DNA, 1515 bp, Methylophilus methylotrophus
  CDS (1)..(1515)
atgactacaaaaaatgcggatattggattggtaggattggcagtgatgggccaaaacctggcgcttaacattgctgatcacggctataccatcgcggtctataaccgtgatccgaaaaaaatgctgaacttcatcgaggagtgcaaaaagaacgagccttcccacgagcgtgtggtgggtcatgccgatctggcgtcttttgtattgagcatcaagcgtcctcgcaagattgtgttgctggtaaaagccggtagcgcgaccgatgtgacgattaacgccttgctgcctttcctggagcagggcgacattattattgatggcggtaatgcgctgtggactgacaccatccgccgtgaaaaagagctggccgccaaaggcattgagtttattggctcaggtgtttccggtggcgaaaccggtgcccgtttcggcccatcgctgatgccttcaggcacgcgcaaggcctgggcaagcctggagcccatctggcgtgatattgctgccaaggtggatcctgtcaccggtacgccattagaaggcggcgctcccggcaaaccggtagaaggcggcttttcctgtgctgaatatatcggcccggatggcgcaggtcactatgtgaaaatggtgcacaacggtatcgaatacatcgatatgcaattgatctgcgaagcctactggctcatgaaaaacctgctcggcatgccagcagacgaaattggtaaagtatttgccgagtggaacaagggcgagctgtccagcttcctgattgaaatcacggcagatatcctgcaacagaaagacccatcaggcaaaggtttcctggtcgacaatattctggatacagcaggccagaagggtaccggccagtggacggccgccaacgcgcttgaactgggcgcacctgctaacgcgattgcagcggcggtgtatgcgcgtgccttgtccagcttgaaagaggagcgtgtagaagcgagcaagatcctcaaggggccagccattgtgcaagaaaaagacaaggcaggtattattgaggcgatcagaaatgctttgtattgctccaaaatctgcgcttacgcacaaggcttccagctcatcgacaaagcgcaggtggcttacaactggaaactcaacttcggtgagattgcccagatctggcgtggtggttgtatcatccgagcccgcttcctgcaaaaaatcactgatgcttacgcattgaactcacgtttgaaaaatctgatgctggacccttatttcacaaatgccatgaacgaaggtcaggctggctggcgtaaagtgattgcgctggcagtgaccaatggtatccccgcgcaaggttttgctgcggcgctggcttactacgatggttacagaagcgctgatttgccagctaacttactgcaaggccagcgtgactactttggcgcacatacttatgagcgtaaagaccagccacgtggccagttcttccacctcgattggccagaagcaggtcgtccgcaattgacgattgaatag

SEQ ID NO: 42 - protein, 504 aa, Methylophilus methylotrophus
MTTKNADIGLVGLAVMGQNLALNIADHGYTIAVYNRDPKKMLNFIEECKKNEPSHERVVGHADLASFVLSIKRPRKIVLLVKAGSATDVTINALLPFLEQGDIIIDGGNALWTDTIRREKELAAKGIEFIGSGVSGGETGARFGPSLMPSGTRKAWASLEPIWRDIAAKVDPVTGTPLEGGAPGKPVEGGFSCAEYIGPDGAGHYVKMVHNGIEYIDMQLICEAYWLMKNLLGMPADEIGKVFAEWNKGELSSFLIEITADILQQKDPSGKGFLVDNILDTAGQKGTGQWTAANALELGAPANAIAAAVYARALSSLKEERVEASKILKGPAIVQEKDKAGIIEAIRNALYCSKICAYAQGFQLIDKAQVAYNWKLNFGEIAQIWRGGCIIRARFLQKITDAYALNSRLKNLMLDPYFTNAMNEGQAGWRKVIALAVTNGIPAQGFAAALAYYDGYRSADLPANLLQGQRDYFGAHTYERKDQPRGQFFHLDWPEAGRPQLTIE

SEQ ID NO: 43 - DNA, 21 bp, Artificial
  primer
cgccagccaggacagaaatgc

SEQ ID NO: 44 - DNA, 22 bp, Artificial
  primer
agcccaagaaaaaccgctggac

SEQ ID NO: 45 - DNA, 31 bp, Artificial
  primer
gggcctgcagggtttacccgcttgatttacc

SEQ ID NO: 46 - DNA, 29 bp, Artificial
  primer
gggtctagatgcattatcgcagtgcgata

SEQ ID NO: 47 - DNA, 57 bp, Artificial
  Synthetic DNA
aattcgagctcggtacccggggatcctctagagtcgacctgcaggcatgcaagctta

SEQ ID NO: 48 - DNA, 57 bp, Artificial
  Synthetic DNA
gatctaagcttgcatgcctgcaggtcgactctagaggatccccgggtaccgagctcg

SEQ ID NO: 49 - DNA, 30 bp, Artificial
  PCR primer
ctatgatcatttgcctggcggcagtagcgc

SEQ ID NO: 50 - DNA, 31 bp, Artificial
  PCR primer
cttagatctcaaaaagagtttgtagaaacgc

SEQ ID NO: 51 - DNA, 34 bp, Artificial
  primer
gctctagaaagttaggcatctcatccaggggagt

SEQ ID NO: 52 - DNA, 32 bp, Artificial
  primer
gctctagaacaatctgctgctcaacaagcccc